SEQUENCE LISTING (40 sequences)

SEQ ID NO: 1 - protein, 540 aa, human [sequence omitted]

SEQ ID NO: 2 - protein, 18 aa, human
PNATSLHVPHSQEGNSTS

SEQ ID NO: 3 - DNA, 20 bp, human
acccagaggacagcaatgac

SEQ ID NO: 4 - DNA, 20 bp, human
cagagctccatcttcagcct

SEQ ID NO: 5 - DNA, 20 bp, human
tccagggagagaacatttgg

SEQ ID NO: 6 - DNA, 20 bp, human
agtaccagggagcgacttga

SEQ ID NO: 7 - DNA, 21 bp, human
cgatatgcacaattgtctcca

SEQ ID NO: 8 - DNA, 20 bp, human
ttccttaagagctggactgg

SEQ ID NO: 9 - DNA, 20 bp, human
tctccaacatgtcccatcac

SEQ ID NO: 10 - DNA, 20 bp, human
atggaagtcgagccacatct

SEQ ID NO: 11 - DNA, 20 bp, human
ctcaggtccagcttctccag

SEQ ID NO: 12 - DNA, 18 bp, human
ccttctgcctaacggcac

SEQ ID NO: 13 - DNA, 20 bp, human
gcaggccaggtttttaatga

SEQ ID NO: 14 - DNA, 19 bp, human
tgtaccgccagttcaccac

SEQ ID NO: 15 - DNA, 22 bp, human
gacctgtacttttctactggcg

SEQ ID NO: 16 - DNA, 21 bp, human
gctgtcactccacaaatgaag

SEQ ID NO: 17 - DNA, 20 bp, human
atgtgctggcgttttcagag

SEQ ID NO: 18 - DNA, 17 bp, human
aggccagctggagcgtc

SEQ ID NO: 19 - DNA, 20 bp, human
ccaggagtccagcagatgat

SEQ ID NO: 20 - DNA, 20 bp, human
tgtctcggcatagttctgga

SEQ ID NO: 21 - DNA, 20 bp, mouse
aagaggctctgtgggactga

SEQ ID NO: 22 - DNA, 20 bp, mouse
taccaccttggccagtaagg

SEQ ID NO: 23 - DNA, 20 bp, mouse
aggagcaagatgcaggaaaa

SEQ ID NO: 24 - DNA, 20 bp, mouse
caccttcgtgctgttcttca

SEQ ID NO: 25 - DNA, 20 bp, human
cacgagttcgtggaagaggt

SEQ ID NO: 26 - DNA, 20 bp, human
ttcttcgtgcagatgtggag

SEQ ID NO: 27 - DNA, 20 bp, human
ggggatctaaggtggcattt

SEQ ID NO: 28 - DNA, 18 bp, human
ccgcctgcttcagtctgt

SEQ ID NO: 29 - DNA, 20 bp, rat
tggtcagatgaagggtgtga

SEQ ID NO: 30 - DNA, 20 bp, rat
ccggtaaataaccaggcaga

SEQ ID NO: 31 - DNA, 20 bp, rat
gcggtcaacctctctactgc

SEQ ID NO: 32 - DNA, 20 bp, human
gcaccagagcactttccttc

SEQ ID NO: 33 - DNA, 20 bp, human
gcaacagaggatgacgatga

SEQ ID NO: 34 - DNA, 20 bp, human
ggagtacctgaaccccaaca

SEQ ID NO: 35 - DNA, 19 bp, human
gctgcacctcgatcatctg

SEQ ID NO: 36 - DNA, 17 bp, human
ctggctatgcgtgctgg

SEQ ID NO: 37 - DNA, 20 bp, human
gtaacccgttgaaccccatt

SEQ ID NO: 38 - DNA, 20 bp, human
ccatccaatcggtagtagcg

SEQ ID NO: 39 - DNA, 20 bp, human
gcctagcagccgacttagaa

SEQ ID NO: 40 - DNA, 20 bp, human
aaatcacatcgcgtcaacac

(19) United States
(12) Patent Application Publication
(10) Pub. No.: US 2016/0169918 A1
(43) Pub. Date: Jun. 16, 2016

(54) Methods of Diagnosis and Treatment for Pulmonary Arterial Hypertension

(71) Applicant: THE REGENTS OF THE UNIVERSITY OF CALIFORNIA, Oakland, CA (US)

(72) Inventors: Fiona Murray, San Diego, CA (US); Paul Insel, La Jolla, CA (US); Nakon Aroonsakool, Bonita, CA (US); Daniel McDonald, Chula Vista, CA (US); Ohmin Kwon, La Palma, CA (US)

(73) Assignee: THE REGENTS OF THE UNIVERSITY OF CALIFORNIA, Oakland, CA (US)

(21) Appl. No.: 14/907,639

(22) PCT Filed: Jul. 31, 2014
(86) PCT No.: PCT/US2014/049147

§ 371 Date: Jan. 26, 2016

Related U.S. Application Data
(60) Provisional application No. 61/860,503, filed on Jul. 31, 2013.

(51) Int. Cl. G01N 33/74; C07K 16/28

(52) U.S. Cl. CPC G01N 33/74; C07K 16/28; G01N 2800/12; G01N 2333/726; G01N 2800/321; C07K 2317/76

(57) ABSTRACT

Diagnostic and therapeutic agents for pulmonary arterial hypertension (PAH) are provided. Circulating microparticles (MPs) and/or the expression level of GPR75 are significantly increased in PAH-PASMC, but not normal PASMC, thus, providing a non-invasive diagnostic method for PAH. Furthermore targeting MPs and/or GPR75 with specific antibodies, inverse agonists or antagonists is a strategy to treat PAH.

### CROSS-REFERENCE TO RELATED APPLICATIONS

This patent application is a claims priority benefit of U.S. Provisional Application No. 61/860,503 filed on Jul. 31, 2013, which is incorporated herein by reference in its entirety.

### STATEMENT OF GOVERNMENT INTEREST

This invention was made with Government support under Grant No. HL091061 awarded by the National Institutes of Health. The Government has certain rights in the invention.

### SEQUENCE LISTING

The instant application contains a Sequence Listing which has been submitted in ASCII format via EFS-Web and is hereby incorporated by reference in its entirety. Said ASCII copy, created on Jul. 31, 2014, is named PCT_Seq_Listing.txt and is 12,288 bytes in size.

### FIELD OF THE INVENTION

In embodiments, the invention relates generally to diagnostic and therapeutic agents in pulmonary arterial hypertension.

### BACKGROUND

Pulmonary arterial hypertension (PAH) is associated with increased vascular resistance linked to sustained contraction and enhanced proliferation of pulmonary arterial smooth muscle cells (PASMCs); abnormal tone, remodeling in the pulmonary vasculature and inflammation contribute to the progression of the disease. The maintenance of the normally low vascular resistance, pressure and tone in the pulmonary circulation is dependent on the interaction of circulating and locally produced vasomodulatory regulators, many of which act via receptor-mediated signaling pathways. PAH can occur secondary to a number of diseases, such as connective tissue disease (secondary PAH, SPAH), which can be the result of sporadic or familial genetic mutation, or have an unknown cause. PAH has a poor prognosis and remains difficult to treat because few pulmonary-selective vasodilators are available. Current therapy for PAH includes prostanoids, endothelin antagonists, calcium-channel blockers and phosphodiesterase-5 inhibitors. These drugs are not effective in all patient populations, can be cumbersome to use and mortality rate for PAH is still high.

G protein-coupled receptors (GPCRs) are guanine nucleotide exchange factors for heterotrimeric G-proteins, whose α and βγ subunits dissociate and regulate effectors. Gαₛ stimulates adenylyl cyclase, and Gαᵢ inhibits adenylyl cyclase. GPCRs are the largest receptor family (˜3% of genome) and are the largest class of and attractive drug targets in disease since they are expressed on the plasma membrane and are tissue specific.

GPR75 is an orphan G protein coupled receptor that has been mapped to chromosome 2p16 (an orphan GPCR is a GPCR whose endogenous ligand has not yet been identified). The GPR75 gene encodes the 540 amino acid protein (approximately 78 kDa), which is highly expressed in human retinal pigment epithelium and in brain sections. The full-length amino acid sequence of GRP75 is represented by SEQ ID NO: 1. Due to its high expression in the retina GPR75 is also termed retinal GPCR. Analysis of GPR75 has revealed, that although variations in this gene does not underlie Doyne's honeycomb retinal dystrophy and Malattia Leventinese phenotypes, it may be a candidate gene for age-related macular degeneration.

It has been proposed that Regulated upon Activation, Normal T-cell Expressed, and Secreted (RANTES) Chemokine Ligand 5 (CCL5) and Neuropeptide Y may be ligands for GPR75³. Upon treatment with RANTES, an increase in inositol triphosphate (IP₃), and stimulation of Ca²⁺ mobilization has been seen in HEK293 cells overexpressing GPR75. Treatment of GPR75-HEK293 cells with U73122 (a PLC inhibitor) has been found to block the RANTES-mediated increase in Ca²⁺ mobilization. These findings suggest that GPR75 may couple to Gαq/11. Sequence-structure based phylogeny predicted that Neuropeptide Y may be a potential ligand for GPR75. Although GPR75 has previously been shown to be highly expressed in the brain and the eye, no published data exists regarding the possible role of GPR75 in PASMCs or in PAH. Levels of RANTES are increased in the lungs of patients with PAH. Neuropeptide Y stimulates proliferation of human PASMCs.

### SUMMARY

Embodiments provide for novel diagnostic and therapeutic agents for treating PAH. More specifically, using an unbiased approach, a G protein-coupled (GPCR) RT-PCR array (Life Technologies), the invention pertains to GPCRs that are abundantly and uniquely expressed in pulmonary artery smooth muscle cells (PASMCs). Data obtained from the GPCR arrays show that human PASMCs express >135 GPCRs, a substantial number of which (e.g., one quarter) may be orphan GPCRs, i.e., ones without known physiological agonists. As described in embodiments herein, GPCR expression has been found to correlate with function (e.g., of Gs-coupled GPCRs with formation of cAMP, a second messenger generated by the receptors, and with a functional response to receptor activation, inhibition of cellular proliferation), further demonstrating that physiologically relevant GPCRs may be identified using methods disclosed herein.

In certain embodiments, the invention provides that the GPCR arrays using mRNA from PASMC isolated from patients with pulmonary arterial hypertension (PAH) showed that PAH (both IPAH and SPAH) is associated with an increase (>2-fold) in the expression of 41 GPCRs and the most significant increase was in the expression an orphan receptor, namely GPR75. In certain embodiments, the invention provides that the expression of GPR75 is significantly increased in PAH-PASMCs from patients and animal models of PAH, e.g., chronic hypoxic (CH) mouse and rat models and monocrotaline (M) rat models, while GPR75 expression appears to be absent in normal PASMCs. In some embodiments, patients having PAH have higher number of circulating microparticles (MPs), as indicated by higher protein levels, and GPR75 may further be expressed by and detected on the MPs, such that MPs of patients with PAH may have greater expression of MPs and of GPR75.

In some embodiments, the invention provides a non-invasive method for diagnosing PAH by determining an amount circulating microparticles (MPs) or an expression level of GPR75 in a biological sample of a subject, such as a fluid or tissue. An increased level of MPs and/or GPR75 expression in the biological sample of the subject is indicative of PAH. In certain embodiments, the biological sample of the subject may be any bodily fluid including, but not limited to, blood, plasma, or serum; or a bodily tissue including, but is not limited to, lung cells or tissue, or heart cells or tissue.

In certain embodiments, GPR75, a previously unknown cell surface receptor, can be used as a therapeutic target for patients with PAH. Because GPR75 is highly expressed in PAH-PASMCs but not normal PASMCs, certain embodiments provide that GPR75, a cell surface receptor of previously unknown function, is a therapeutic target for patients with PAH. Embodiments also provide for methods of treating PAH by targeting or binding GPR75 with inhibitors, such as specific antibodies, inverse agonists, or antagonists, to treat PAH by inhibiting PASMC proliferation, pulmonary arterial pressure and effects of inflammatory cytokines, such as RANTES. In certain embodiments, monoclonal antibodies targeted to the N-terminal of GPR75 may be used to increase cAMP and/or inhibit intracellular Ca²⁺ and inhibit DNA synthesis, and thus the proliferation, of PAH-PASMCs. For example, an anti-GPR75 antibody may be used, which may be generated against a conserved sequence in the N-termini of the GPR75 protein by cyclic peptide methodology, which is thought to result in higher titer and more specific antibodies.

In further embodiments, the immunogen used to generate the anti-GPR75 antibody may be the synthetic cyclic peptide (PNATSLHVPHSQEGNSTS (SEQ ID NO: 2)-amide). The full-length amino acid sequence of GPR75 (SEQ ID NO: 1) is available under accession number AAH67475 from the National Institutes of Health. It will be readily appreciated by persons skilled in the art that GPR75 has many immunogenic portions that can be used to routinely generate alternative antibodies. In certain embodiments, the anti-GPR75 antibody is a human monoclonal antibody or a monoclonal antibody that is suitable for humanization.

In certain embodiments, an antagonist or inverse agonist to GPR75 beneficially inhibits PAH-PASMC proliferation and decreases pulmonary artery pressure. In certain embodiments, using any art-accepted animal model of PAH (e.g., a chronic hypoxic (CH) rat and mouse model or monocrotaline-treated rats) or appropriate cells (e.g., HEK or PASMCs), agents that inhibit GPR75, such as antibodies, inverse agonists or antagonists directed to GPR75) can be screened and developed to be used in preventing or reversing remodeling of the pulmonary artery and reducing pulmonary artery pressure. Therefore, provided is a direct therapeutic approach for treating PAH by targeting GPR75, either with an antagonist or, inverse agonist, including the use of antibody against GPR75, alone or in combination with currently approved therapies, to inhibit PASMC proliferation, decrease pulmonary arterial remodeling, and thereby decrease pulmonary arterial pressure in PAH.

In certain embodiments, a method for diagnosing or treating pulmonary arterial hypertension (PAH) in a subject comprises obtaining a biological sample from said subject, and measuring an expression level of GPR75 in the sample of the subject, wherein an increased expression level of GPR75 in the sample of the subject as compared to a reference level of GPR75 in a control sample provides a diagnosis, an indication for treatment of PAH or means to monitor therapy of PAH in the subject. In such embodiments, the biological sample may be selected from the group including blood, plasma, serum, lung cells or tissues, and heart cells or tissues. The cells may be pulmonary arterial smooth muscle cells (PASMCs).

In certain embodiments, a method for diagnosing or treating pulmonary arterial hypertension (PAH) in a subject in need thereof may comprise obtaining a biological sample from said subject, and measuring an amount of circulating microparticles (MPs) in the sample of the subject, wherein an increased amount of MPs as compared to a reference amount of MPs in a control sample provides a diagnosis or an indication for treatment of PAH in the subject. In such embodiments, the measuring of the amount of circulating MPs in the sample of the subject comprises detecting GPR75 that is expressed on said MPs.

Embodiments also relate to a kit for diagnosing pulmonary arterial hypertension (PAH) in a subject in need thereof that comprises: (a) a capture reagent comprising one or more detectors specific for binding to circulating microparticles (MPs) and/or to GPR75 expressed thereon; (b) a detection reagent specifically reactive with the capture reagent; and (c) instructions for using the kit for diagnosing and providing an indication of a treatment of PAH in said subject when an increased amount of the circulating MPs or an expression level of the GPR75 is detected in a bodily sample of the subject bodily sample as compared to a reference amount of circulating MPs or a reference expression level of GPR75 in a control sample.

In certain embodiments, a method for treating PAH comprises: administering to a subject thereof a pharmaceutical composition comprising a therapeutically effective amount of an agent that inhibits GPR75. The agent may be a GPR75 inverse agonist or antagonist that interacts with the GPR75 expressed by pulmonary arterial smooth muscle cells (PASMCs) of the subject so as to inhibit signaling or function, or an mRNA, DNA, or protein expression level, of the GPR75. In some embodiments, the agent may regulate a second message signaling pathway associated with the GPR75. In further embodiments, the agent may be an anti-GPR75 antibody, and the anti-GPR75 antibody may be a human or humanized monoclonal antibody.

In certain embodiments, a method for treating PAH comprises administering to a subject in need thereof a pharmaceutical composition comprising a therapeutically effective amount of an agent that inhibits circulating microparticles (MPs) or GRP75 expressed thereon. In such embodiments, administration of the agent reduces an amount of circulating MPs that are associated with PAH and/or an expression level of GPR75 expressed on MPs associated with PAH. The agent may be anti-GPR75 antibody, and the anti-GPR75 antibody may be human or humanized.

### BRIEF DESCRIPTION OF THE DRAWINGS

FIG. 1 is a schematic representation of the cyclic AMP (cAMP) pathway. Upon ligand binding of a Gαₛ-coupled GPCR, the heterotrimeric GDP-bound G protein exchange GDP for GTP on the Gαₛ subunit, thereby promoting subunit dissociation and activation of AC, which catalyzes cAMP formation from ATP. cAMP is degraded (hydrolyzed) by PDEs to 5′-AMP. cAMP activates the downstream effectors, PKA and EPAC, which lead to vasodilation and inhibition of proliferation of PASMC. There are multiple ways to increase intracellular levels of cAMP in PASMCs: (1) activate Gαₛ-coupled GPCRs; block Gαᵢ-coupled GPCRs, (2) increase AC expression or activity, (3) inhibit PDE expression or activity, and/or (4) activate PKA and EPAC or proteins that those effectors regulate.

FIG. 2 is a schematic diagram of G protein-coupled receptor (GPCR)-dependent signaling in pulmonary artery smooth muscle cells (PASMC). Binding of agonist ligands to a GPCR catalyzes the exchange of bound GDP for GTP on the Gα-subunit, causing it to dissociate from the Gβγ-subunits. The Gαₛ-subunit activates adenylyl cyclase (AC), which facilitates the conversion of ATP to 3′,5′-cyclic adenosine monophosphate (cAMP). Cyclic AMP promotes vasodilation and decreases proliferation of PASMCs. GPCRs that activate Gαᵢ inhibit AC activity and decrease intracellular cAMP; Gα_{q/11} stimulates membrane-bound phospholipase C β (PLCβ), which cleaves phosphatidylinositol 4,5-bisphosphate (PIP2) into inositol 1,4,5-triphosphate (IP₃) and diacylglycerol (DAG), which lead to increased intracellular Ca²⁺ and protein kinase C activity, respectively Gα_{12/13} regulates Ras homolog gene family, member A (RhoA); Gβγ can activate phosphoinositide 3-kinase γ (PI3Kγ) and also PLCβ, all leading to PASMC vasoconstriction and increased proliferation.

FIG. 3 shows microarray ΔCₜ values compared to independent real-time PCR ΔCₜ Values (r²=0.70).

FIG. 4A is a correlation graph comparing mRNA expression with cAMP accumulation in Gαₛ-coupled GPCRs in PASMC (r²=0.31).

FIG. 4B shows cAMP accumulation induced by GPCR-selective agonists correlates with their ability to decrease PASMC proliferation (r²=0.90).

FIG. 4C shows: (a) VIPR1 being expressed on the membrane in Ctrl-PASMCs; (b) vasoactive intestinal peptide (VIP) increasing cAMP levels in Ctrl-PASMCs in a concentration-dependent manner; and (c) VIP decreasing proliferation (measured as DNA synthesis using [³H]thymidine) of Ctrl-PASMCs in a concentration-dependent manner.

FIG. 4D shows: (a) OXTR being expressed on the membrane in Ctrl-PASMCs; (b) oxytocin decreasing forskolin-stimulated cAMP levels in Ctrl-PASMCs in a concentration-dependent manner; and (c) oxytocin increasing proliferation of Ctrl-PASMCs in a concentration-dependent manner.

FIG. 5A shows Venn Diagrams depicting increases in mRNA expression (>2-fold) in (IPAH)- and secondary pulmonary arterial hypertension (SPAH)-PASMCs compared to Control (Ctrl)-PASMCs FIG. 5B shows Venn Diagrams depicting decreases in mRNA expression (<0.5-fold) in IPAH- and SPAH-PASMCs compared to Ctrl-PASMCs.

FIG. 6A shows GPR113 protein expression in PAH-PASMCs compared to Ctrl-PASMCs (a representative blot).

FIG. 6B shows GPR113 mRNA expression in Chronic hypoxic (CH) rat lungs (n=4) compared to control (n=4). Cₜ values normalized to 18S. The change in GPR113 mRNA expression is statistically significant (P<0.05), according to Student's t-test.

FIG. 6C shows fold-change in GPR113 mRNA expression normalized to 18S RNA in CH rat lungs (n=4) compared to control (n=4). The change in GPR113 mRNA expression is statistically significant (P<0.05), according to Student's t-test.

FIG. 6D shows GPR113 mRNA expression in monocrotaline (MCT)-treated rat lungs (n=3) compared to control (n=3). Cₜ values normalized to 18S RNA. The change in GPR113 mRNA expression is statistically significant (P<0.01), according to Student's t-test.

FIG. 6E shows fold-change in GPR113 mRNA expression normalized to 18S RNA in MCT-treated rat lungs (n=3) compared to control lungs (n=3). The change in GPR113 mRNA expression is statistically significant (P<0.01), according to Student's t-test.

FIG. 7A shows GPR75 protein expression in Ctrl-, SPAH-, and IPAH-PASMCs (a representative blot).

FIG. 7B shows immunofluorescence images that demonstrate increased GPR75 expression in IPAH-PASMCs compared to control. Secondary antibody was tagged with FITC for visualization of GPR75 (green gray scales). DAPI was used for visualization of the nucleus (blue gray scales). Cells were not permeabilized. The image was taken at 60× magnification.

FIG. 7C shows GPR75 protein expression in CH mouse lungs compared to control lungs (a representative blot).

FIG. 7D shows GPR75 protein expression in CH rat lungs compared to control lungs (a representative blot).

FIG. 7E shows GPR75 protein expression in MCT-treated rat lungs compared to control lungs (a representative blot).

FIG. 7F shows GPR75 mRNA expression in CH rat lungs (n=4) compared to control lungs (n=4). Cₜ values normalized to 18S RNA. The change in GPR75 mRNA expression is statistically significant (P<0.05), according to Student's t-test.

FIG. 7G shows fold-change in GPR75 mRNA expression normalized to 18S RNA in CH rat lungs (n=4) compared to control lungs (n=4). The change in GPR75 mRNA expression is statistically significant (P<0.05), according to Student's t-test.

FIG. 7H shows GPR75 mRNA expression in MCT-treated rat lungs (n=3) compared to control lungs (n=3). Cₜ values normalized to 18S RNA. The change in GPR75 mRNA expression is statistically significant (P<0.05), according to Student's t-test.

FIG. 7I shows fold-change in GPR75 mRNA expression normalized to 18S RNA in MCT-treated rat lungs (n=3) compared to control (n=3). The change in GPR75 mRNA expression is statistically significant (P<0.05), according to Student's t-test.

FIG. 7J shows GPR75 protein expression in control (n=2) and CH (n=2) rat heart left ventricle (LV) and right ventricle (RV).

FIG. 7K shows cAMP accumulation in Ctrl-PASMCs (n=2) and PAH-PASMCs (n=3) in the presence and absence GPR75 antibody (1 μg/mL). All cells received 10 min forskolin (10 μM) stimulation. There is no significant change in cAMP accumulation in Ctrl-PASMCs treated with IgG control or GPR75 antibody, but in PAH-PASMCs, the GPR75 antibody increased cAMP accumulation. This increase in cAMP accumulation is statistically significant (P<0.05), according to Student's t-test. The decrease in cAMP in IgG-treated PAH-PASMCs compared to Ctrl-PASMCs is statistically significant (P<0.01), according to Student's t-test.

FIG. 7L shows DNA synthesis and proliferation ([³H]Thymidine incorporation) of Ctrl-PASMC (n=4) in the presence of IgG control, cytoplasmic-domain targeted or N-terminal-targeted GPR75 antibody (all 1 μg/mL).

FIG. 7M shows DNA synthesis and proliferation ([³H]Thymidine incorporation) of IPAH-PASMCs (n=4) in the presence of IgG control, cytoplasmic domain-binding GPR75 antibody, and N-terminal binding GPR75 antibody (all (1 μg/mL). The N-terminal GPR75 antibody, but not the cytoplasmic domain-targeted antibody, decreases proliferation compared to the IgG control. This decrease in proliferation is statistically significant (P<0.05), according to Student's t-test.

FIG. 7N shows transfection of GPR75 plasmid (2.5 μg) into HEK 293 cells. Fold-change in GPR75 expression normalized to GAPDH compared to empty vector control. Maximal fold-change occurred 72-hr after transfection.

FIG. 7O shows cAMP accumulation in GPR75 vector-transfected HEK 293 cells (n=3) compared to empty vector-transfected cells (n=3). Cells transfected with the GPR75 construct had statistically significant (P<0.01, according to Student's t-test) lower basal levels of cAMP and lower cAMP levels if incubated with forskolin (FSK, 10 μM) for 10 μM) min.

FIG. 7P shows the effect of N-terminal-targeted GRP75 polyclonal antibody on forskolin (FSK)-stimulated (10 μM FSK, 10 min) cAMP accumulation in GPR75 vector-transfected HEK 293 cells (n=3) compared to empty vector-transfected cells (n=3). Cells transfected with the GPR75 construct had statistically significant (P<0.01, according to Student's t-test) lower FSK-stimulated cAMP levels, but their cAMP levels were restored to that of IgG-treated empty vector-transfected (control) cells by incubation with the N-terminal-direction GPR75 polyclonal antibody.

FIG. 7Q shows [³H]Thymidine incorporation in HEK 293 cells expressing GPR75 (n=6). Such cells show an increase in proliferation compared to empty vector-transfected cells (n=6). This increase in proliferation is statistically significant (P<0.01), according to Student's t-test.

FIG. 7R shows a Western Blot of GPR75 expressed HEK 293 cells.

FIG. 7S shows deglycosylation of GPR75 in CH Rat lung, SPAH-PASMCs, and GPR75-expressing (O/E) HEK 293 cell protein lysate treated without (−) and with (+) PNGase F added (1 μL at 500,000 U/mL).

FIG. 8 is a schematic representation of a potential GPR75 signal transduction pathway. The left panel shows a PAH-PASMC without use of a blocking antibody, RANTES activation of GPR75, and coupling to Gαᵢ- and Gα_{q/11}. The outcome of this response is vasoconstriction and increased PASMC proliferation. The right panel shows a PAH-PASMC treated with N-terminal binding anti-GPR75 antibody. This antibody will bind and block the receptor, thus blunting the activation from RANTES. In turn, the Gαᵢ- and Gα_{q/11}-coupled pathways will not be activated, leading to higher intracellular cAMP levels and decrease in intracellular Ca²⁺ thus, decreased proliferation of PASMC and less vasoconstriction.

FIG. 9A is an image of a lung slice from a CH Rat showing vascular remodeling (a) compared to control lung (b). The image was taken at 40× magnification.

FIG. 9B is a pCMV6-Entry Vector schematic taken from OriGene Technologies, Inc.

FIG. 9C is a GPR75 Restriction Map (from New England Biolabs, Inc).

FIG. 9D is a Restriction Digest with Bgl II confirming GPR75 DNA insertion in a pCMV6-Entry Vector.

FIG. 9E is a Western Blot of GPR75 overexpression in PASMCs.

FIG. 10 shows that circulating microparticles (MPs) increase in pulmonary arterial hypertension (PAH) and express more GPR75. The MPs were isolated from the blood of PAH patients and control subjects. Protein content, determined by Bradford analysis, is greater in MPs from PAH patients than in controls (n=4). Western blot of MP protein lysates from 2 PAH patients and controls show GPR75 protein is detected in these lysates and that MPs from the PAH patients have more GPR75.

FIG. 11 is an image of immunofluorescence showing increased GPR75 expression in the smooth muscle layer of pulmonary arteries of rats with chronic-hypoxia-induced PAH compared to controls. Lung slices (10 microns thick) stained for α-smooth muscle actin (α-SMA, green grayscales) and GPR75 (red grayscales) show their co-localization (Merge column) in pulmonary artery smooth muscle of the hypoxic rat lungs.

FIG. 12 shows mouse-derived ascites fluid that contains an N-terminal directed GPR75 monoclonal antibody having increased cAMP accumulation in PAH-PASMCs, but not in Control-PASMCs.

FIG. 13A shows mouse-derived ascites fluid that contains an N-terminal directed GPR75 monoclonal antibody having increased cAMP accumulation and FIG. 13B shows decreased DNA synthesis of GPR75-expressing HEK293 cells.

FIG. 14 shows increased cAMP accumulation in PAH-PASMCs but not in Control-PASMCs. Mouse-derived ascites fluid that contain N-terminal-directed monoclonal antibody (mAb, 1 μg/mL) from 3 mice (C248, C816 and C818) or conditioned media from the cell culture of each individual hybridoma increase cAMP accumulation of PAH-PASMCs, but not Control (Ctrl)-PASMCs, relative to mouse IgG.

### DETAILED DESCRIPTION OF EMBODIMENTS

Embodiments provide for novel diagnostic and therapeutic agents for treating PAH.

In certain embodiments, a method for diagnosing or treating pulmonary arterial hypertension (PAH) in a subject comprises obtaining a biological sample from said subject, and measuring an expression level of GPR75 or GPR113 in the sample of the subject, wherein an increased expression level of GPR75 or GPR113 in the sample of the subject as compared to a reference level of GPR75 or GPR113 in a control sample provides a diagnosis or an indication for treatment of PAH in the subject. In such embodiments, the biological sample may be selected from the group consisting of blood, plasma, serum, lung cells or tissues, and heart cells or tissues. The cells may be pulmonary arterial smooth muscle cells (PASMCs). The step of obtaining a biological sample encompasses a broad range of physical activity, including drawing a bodily fluid or tissue from a patient to manipulating a previously drawn sample from the patient while conducting a diagnostic assay, for example.

In certain embodiments, a method for diagnosing or treating pulmonary arterial hypertension (PAH) in a subject in need thereof may comprise obtaining a biological sample from said subject, and measuring an amount of circulating microparticles (MPs) in the sample of the subject, wherein an increased amount of MPs as compared to a reference amount of MPs in a control sample provides a diagnosis, an indication for treatment of PAH or a means to monitor PAH in the subject. In such embodiments, the measuring of the amount of circulating MPs in the sample of the subject comprises detecting GPR75 that is expressed on said MPs. Measurements/data obtained according to diagnostic methods described herein may be used routinely formulating a range of dosages for use in the subject.

Embodiments also relate to a kit for diagnosing pulmonary arterial hypertension (PAH) in a subject in need thereof that comprises: (a) a capture reagent comprising one or more detectors specific for binding to circulating microparticles (MPs) and/or to GPR75 expressed thereon; (b) a detection reagent specifically reactive with the capture reagent; and (c) instructions for using the kit for diagnosing and providing an indication of a treatment of PAH in said subject when an increased amount of the circulating MPs or an expression level of the GPR75 is detected in a bodily sample of the subject bodily sample as compared to a reference amount of circulating MPs or a reference expression level of GPR75 in a control sample.

Capture and detection of GPR75 can be achieved through a wide variety of detection reagents, including labeled antibodies, protein detection assays and mRNA assays, for example.

In certain embodiments, a method for treating PAH comprises: administering to a subject thereof a pharmaceutical composition comprising a therapeutically effective amount of an agent that inhibits GPR75 or GPR113. The agent may be a GPR75 or GPR113 agonist, inverse agonist or antagonist that interacts with the GPR75 or GPR113 expressed by pulmonary arterial smooth muscle cells (PASMCs) of the subject so as to inhibit signaling or function, or an mRNA, DNA, or protein expression level, of the GPR75 or GPR113. In some embodiments, the agent may regulate second message signaling pathway associated with the GPR75 or GPR113. In further embodiments, the agent may be an anti-GPR75 or anti-GPR113 antibody, and the anti-GPR75 or anti-GPR113 antibody may be a human or humanized monoclonal antibody.

In certain embodiments, a method for treating PAH comprises administering to a subject in need thereof a pharmaceutical composition comprising a therapeutically effective amount of an agent that inhibits circulating microparticles (MPs) or GRP75 expressed thereon. In such embodiments, administration of the agent reduces an amount of circulating MPs that are associated with PAH and/or an expression level of GPR75 expressed on MPs associated with PAH. The agent may be anti-GPR75 antibody, and the anti-GPR75 antibody may be human or humanized. Effective amounts of the composition are those which have the result of at least improving a condition or symptom of a patient with PAH.

An agent administered according to methods described herein may be conjugated to a therapeutic moiety, such as a cytotoxin, a therapeutic agent, or a radioactive metal ion. The conjugates may be used for modifying a given biological response. The agents described herein may be administered in the form of expressible nucleic acids which encode said agents. For instance, the nucleic acid molecules can be constructed from the known coding sequence of GPR75 or GPR113 for RNA interference (RNAi) or be inserted in to vectors and used as gene therapy vectors. Pharmaceutical preparations of the gene therapy vector can include the gene therapy vector in an acceptable diluent, or can comprise a slow release matrix in which the gene delivery vehicle is embedded.

Pharmaceutical compositions may comprise, in addition to the active agent, a wide variety of well-known excipients, diluents and stabilizers as are routinely used in the art of pharmacology. The pharmaceutical compositions may be included in a container, pack, or dispenser, together with instructions for administration.

In practicing methods of the invention, an “effective amount” of an agent in an amount that is necessary to achieve the desired result at prescribed dosages and for periods of time. A therapeutically active amount of GPR75 or GPR113 will be understood by those skilled in the art as modulated by factors such as, e.g., disease state, age, sex, and weight of the patient, and the ability of peptide to elicit a desired response in the patient.

Data obtained from diagnostic methods according to the invention, or from cell culture assays and animal studies, can be used in formulating a range of dosage for use in human subjects. The dosage may vary depending on the form employed and the route of administration utilized. For any compound used in methods of the present disclosure, the therapeutically effective dose can be estimated initially from cell culture assays.

In embodiments, a therapeutically effective amount of protein or polypeptide (i.e., an effective dosage) may range from about 0.001 to 30 mg/kg body weight. The skilled artisan will appreciate that certain factors may influence the dosage required to effectively treat a subject, including (but not limited to) the severity of the disease, disorder, or condition, previous treatments, the general health and/or age of the subject, and presence of any other diseases. Moreover, treatment of a subject with a therapeutically effective amount of an agent (e.g., a protein, polypeptide, or antibody) may include a single treatment or a series of treatments.

Dosage regimens can be adjusted to provide the optimum therapeutic response. For example, several divided doses can be administered daily or the dose can be proportionally reduced as indicated by the exigencies of the therapeutic situation.

### Abbreviations

cAMP=3′,5′-cyclic adenosine monophosphate;
PKA=Protein Kinase A;
EPAC=Exchange Protein directly Activated by cAMP;
PDE=Phosphodiesterase;
FSK=Forskolin;
IBMX=3-isobutyl-1-methylxanthine;
GPCR=G protein-coupled receptor;
PAH=Pulmonary Arterial Hypertension;
SPAH=Secondary Pulmonary Arterial Hypertension;
IPAH=Idiopathic Pulmonary Arterial Hypertension;
CH=Chronic Hypoxic;
MCT=Monocrotaline;
PASMC=Pulmonary Artery Smooth Muscle Cells;
HEK 293 Cells=Human Embryonic Kidney 293 Cells;
PAP=Pulmonary Arterial Pressure;
RANTES=Regulated Upon Activation Normal T-Cell Expressed, and Secreted.

### Pulmonary Arterial Hypertension (PAH)

Pulmonary arterial hypertension (PAH) is characterized by increased pulmonary vascular resistance, in part due to increased proliferation of pulmonary artery smooth muscle cells (PASMCs). Since the second messenger 3′5′-cyclic adenosine monophosphate (cAMP) decreases proliferation of PASMCs, G protein-coupled receptors (GPCRs) coupled to Gαₛ are attractive agents for PAH. In carrying out certain embodiments described herein, TAQMAN human GPCR arrays were used to identify the GPCRs expressed by PASMCs isolated from normal subjects and from patients with PAH. The data revealed that human PASMCs express >135 GPCRs, at least 50 of which regulate cAMP formation. It was therefore found that GPCR expression correlates with function e.g., of Gαₛ-coupled GPCRs with formation of cAMP and inhibition of cell proliferation (a functional response to receptor activation), thus evidencing that physiologically relevant GPCRs had been identified. Experiments relating to PAH-PASMC with GPCR arrays further revealed that PAH (both idiopathic [IPAH] and secondary PAH [SPAH]) is associated with an increase (>2-fold) in the expression of 41 GPCRs. The greatest increase in GPCR expression was of two orphan receptors, namely GPR113 and GPR75, whose expression was absent in normal PASMC. It was also found the mRNA and protein expressions of GPR113 and GPR75 were increased in animal models of PAH. Importantly, treatment of PAH-PASMC with a GPR75 antibody blunted the increased proliferation of PASMC and increased cellular cAMP levels. Taken together, the data summarized herein demonstrates that the GPCR microarray can identify GPCRs that contribute to the physiology of PASMC and can uncover new drug targets, such as GPR75 for PAH—a disease that requires therapies beyond those currently in use.

The pulmonary circulation is a low resistance, low pressure and highly compliant circulation, which allows for free gas exchange. Deoxygenated blood is pumped from the right ventricle through the pulmonary artery, where oxygen diffuses into blood and is exchanged for carbon dioxide in the hemoglobin of the erythrocytes. The oxygen-rich blood returns to the heart via the pulmonary veins to be pumped ultimately from the left ventricles into the systemic circulation. Normal (systolic/diastolic) pulmonary arterial pressure (PAP) is 24/9 mmHg with a mean arterial pressure of 15 mmHg, much lower than the average systolic/diastolic arterial pressures (120/80 mmHg) in the systemic circulation. Abnormal vasoconstriction, pulmonary vascular remodelling and/or thrombosis in situ can lead to an increase in PAP and the development of pulmonary arterial hypertension (PAH), high blood pressure in the pulmonary circulation.

PAH is characterized by a mean PAP of greater than 25 mmHg at rest.¹⁻² PAH can occur secondary to a number of diseases (secondary PAH, SPAH), such as (but not limited to) connective tissue disease, chronic obstructive pulmonary disease, that can be the result of a sporadic or familial genetic mutation or can be primary or idiopathic (IPAH), which have an unknown cause.³ The female to male ratio for IPAH is about 2:1, suggesting that women may be predisposed to the disease.⁴

PAH is associated with increased vascular resistance due to sustained contraction and narrowing of the small pulmonary arteries (PAs): increased proliferation of pulmonary artery smooth muscle cells (PASMC) contributes to remodeling of the PAs. Muscularization of peripheral arteries, medial hypertrophy of muscular arteries (which includes proliferation of fibroblasts and PASMC, endothelial cell swelling, and fragmented elastin), neointima formation (invasion of inflammatory cells), plexiform lesion formation (endothelial channel formation), and loss of small precapillary arteries contributes to the progression of PAH.⁵ Symptoms of PAH include shortness of breath with exercise, difficulty breathing at rest, dizziness and chest pain due to the excessive strain on the heart. The abnormally high pressure in the PA leads to right ventricular hypertrophy, which can ultimately lead to heart failure.

PAH has a poor prognosis and currently no cure. The goal of treating PAH with drugs is to reduce pressure and resistance in the PAs and to increase cardiac output. Current treatments for PAH include anticoagulants, vasodilators, and heart/lung transplantation.⁶⁻⁸ Vasodilators that are currently used clinically are calcium channel blockers, intravenous prostacyclin, inhaled nitric oxide (NO), endothelin receptor antagonists and cyclic nucleotide phosphodiesterase (PDE) 5 inhibitors and a guanylyl cyclase activator. A major issue with the development of drugs for PAH is their lack of specificity for the pulmonary circulation. Vasodilators in the pulmonary circulation also tend to vasodilate the systemic circulation, leading to systemic hypotension. Discovery of pulmonary-selective targets is thus important in the development of future therapies for PAH.

Cyclic AMP (cAMP)

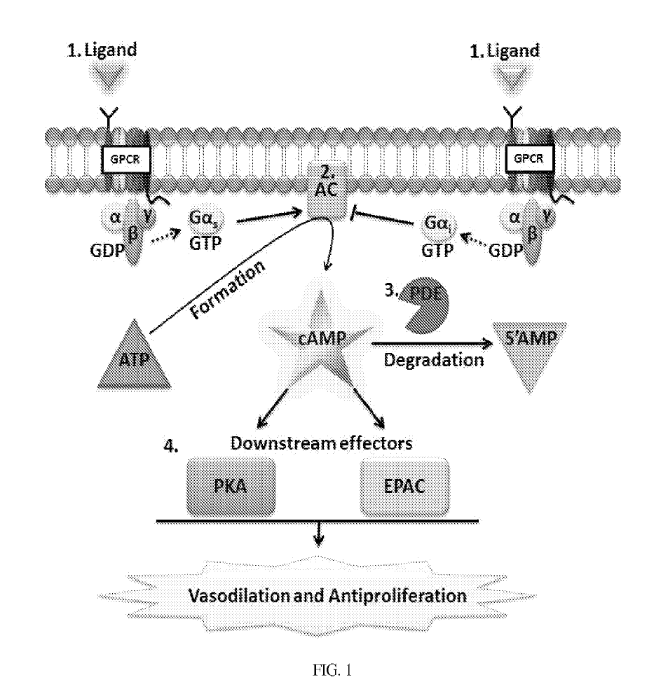

3′,5′-cyclic adenosine monophosphate (cAMP) is a ubiquitous intracellular second messenger that was discovered by Rall and Sutherland in 1958.⁹ Cyclic AMP has many effects: reduces inflammation and systemic blood pressure, can be pro-apoptotic in certain cell types (such as immature lymphoid cells) or anti-apoptotic in other cell types (such as epithelial cells), decreases platelet aggregation, inhibits fibrosis, causes bronchodilation, inhibits PASMC proliferation, and vasodilates the PA.¹⁰⁻¹² An intracellular concentration of cAMP may be determined by activation of G protein-coupled receptors (GPCRs) that stimulate or inhibit the activity of adenylyl cyclases (and thus, the synthesis of cAMP) and by PDEs, which hydrolyze cAMP. Nine membrane-bound isoforms of mammalian ACs have been characterized, each with their own tissue distribution and regulation; AC6, which is Gαₛ-coupled, is highly expressed in PASMCs.^{13,14} Eleven PDEs have been characterized that can hydrolyze cAMP to 5′-AMP, thus reducing its intracellular concentration (FIG. 1). PDE1, PDE3 and PDE4 appear to control cAMP degradation in PASMCs.¹¹ The intracellular level of cAMP and the duration of its signaling can thus be controlled by the balance between formation by ACs and hydrolysis by PDEs.¹⁵⁻¹⁷ cAMP primarily activates two downstream effectors, protein kinase A (PKA) and the exchange protein directly activated by cAMP (EPAC). Both of these effectors contribute to the antiproliferative and vasodilatory effects of cAMP in the PA.

Targeting cAMP in PASMC

As outlined above, cAMP relaxes PA smooth muscle and helps control pulmonary vascular tone.¹⁸⁻²⁰ Increasing cAMP also inhibits PASMC proliferation.²¹⁻²³ A membrane permeable cAMP analog, 8Br-cAMP, reduced the percentage of cells in the S phase of the cell cycle after serum stimulation, by preventing cell cycle progression from G0/G1.^{24,25} It has been proposed that cAMP decreases smooth muscle cell proliferation through both PKA and EPAC activation and the inhibition of mitogenic pathways.²⁶⁻³¹ Stimulation of PKA results in the phosphorylation of a number of proteins, thereby, regulating cellular processes and gene expression, which can produce vasodilation of the PA smooth muscle. PKA can phosphorylate Raf-1 on serines 43 and 621, thus inhibiting p42/p44 mitogen-activated protein kinase (MAPK) activation.^{32,33} Inhibition of the phosphoinositide 3-kinase (PI3K) pathway by cAMP may also play a role in attenuating cell proliferation.³⁰

EPAC-1 and EPAC-2 are cAMP-dependent guanine-nucleotide-exchange factors for the small GTPases Rap1 and Rap2, which are important mediators of cAMP signaling. EPACs have been associated with various cellular processes, such as integrin-mediated cell adhesion and cell-cell junction formation.³⁴ In a vascular injury mouse model, EPAC-1 was shown to be up-regulated during neointima formation and to promote vascular smooth muscle migration.³⁵ These data suggest that EPAC-1 regulates vascular remodeling upon vascular injury. EPAC-1 is decreased in PAH-PASMCs.³¹ Elevation of cAMP in response to β2-adrenergic receptor agonists or prostanoids activates both PKA and EPAC and can induce airway smooth muscle relaxation, inhibit airway smooth muscle proliferation, and modulate cytokine secretion. EPAC induces airway smooth muscle relaxation through inhibition of RhoA and activation of Rac1. EPAC and PKA inhibit airway smooth muscle proliferation and cytokine secretion by signaling to PKB/Akt, p70S6K, ERK1/2, and NF-kB.³¹

There are multiple ways to increase intracellular levels or the function of cAMP in PASMCs and produce vasodilation and decreased proliferation: (1) activate Gαₛ-coupled GPCRs, block Gαᵢ-coupled GPCRs, (2) increase expression or activity of ACs, (3) inhibit PDE expression or activity, and/or (4) activate downstream effectors of cAMP (PKA and EPAC) or by altering the expression or activity of proteins regulated by PKA and EPAC (FIG. 1). GPCRs are attractive drug targets to raise cAMP in PASMC since they 1) localize on the plasma membrane, making them easily accessible to drugs; 2) are the largest receptor family, comprising of 3% of the human genome; 3) are the targets for over 30% of prescribed drugs, and 4) can be tissue-specific, which is beneficial in efforts to selectively target the pulmonary circulation.^{36,37} GPCRs are the most “upstream” component in the signal transduction pathway, thus the targeting of GPCRs benefits from the post-receptor amplification that occurs in this signaling pathway.

### G Protein-Coupled Receptors

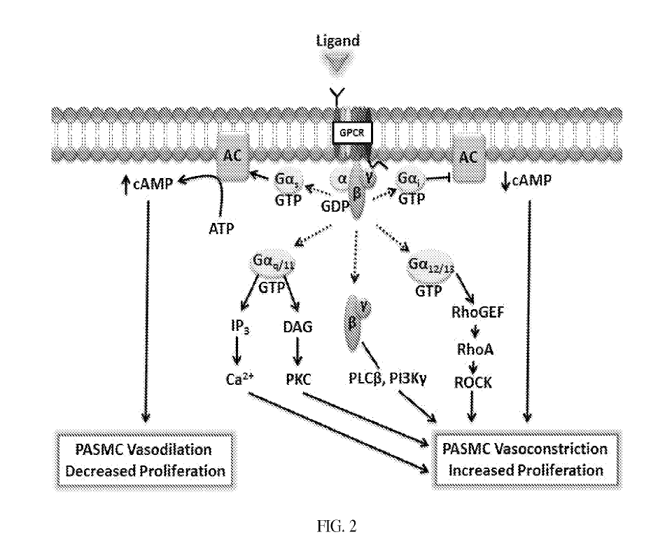

GPCRs comprise a large protein family of 7 transmembrane receptors, which are guanine nucleotide exchange factors for heterotrimeric G proteins. Activation of these receptors occurs when ligands bind to the extracellular domain of the receptor, altering its conformation and in turn, the activity of membrane-bound heterotrimeric guanine nucleotide (G) proteins: guanosine diphosphate (GDP)-bound Gα subunit and a Gβγ complex. Binding of agonist ligands to GPCRs promotes the exchange of the bound GDP for guanosine triphosphate (GTP) on the Gα-subunit, thus facilitating its dissociation from the receptor and the Gβγ heterodimer. The heterotrimeric G proteins are divided into four classes based on their a subunit: Gαₛ, Gαᵢ, Gα_{q/11}, and Gα_{12,13} (FIG. 2). Gαₛ stimulates the activity of AC, which catalyzes the synthesis of cAMP from ATP while Gαᵢ inhibits AC activity and thus decreases cAMP synthesis. Gα_{q/11} stimulates membrane-bound phospholipase C β (PLCβ), which cleaves phosphatidylinositol 4,5-bisphosphate (PIP2) into inositol 1,4,5-triphosphate (IP₃) and diacylglycerol (DAG). Gα_{12/13} regulates Ras homolog gene family, member A (RhoA), a low molecular weight GTPase which influences the actin cytoskeleton. In addition, the Gβγ heterodimer can activate PI3Kγ and also PLCβ.^{38,39}

Unbiased approaches have begun to identify GPCR expression in specific tissues. The quantification of RNA transcripts for 353 non-odorant GPCRs in 41 tissues from mice revealed new roles for a number of GPCRs in various tissues.⁴⁰ Many orphan GPCRs (receptors whose endogenous agonist ligand is not known) and olfactory GPCRs are expressed in tissues, but their function has yet to be determined.⁴¹ Limited data are available regarding GPCR expression in individual cell types. Because the GPCR profile of PASMCs has not been identified, key GPCRs that regulate the pulmonary circulation may have been overlooked. Profiling GPCR expression in PASMCs from control, IPAH and SPAH patients thus has the potential to identify GPCRs that may contribute to the pathophysiology of PAH and that could be novel therapeutic targets for this disease.

Objectives of the discovery effort included:
(1) investigate the mRNA expression of GPCRs in PASMCs through an unbiased approach using a GPCR real-time PCR array
(2) validate results obtained from the arrays by measuring mRNA, and function of highly expressed Gαₛ/Gαᵢ-coupled GPCRs,
(3) investigate if PAH is associated with the altered expression of GPCRs which could be novel targets for the disease

Previously unrecognized GPCRs, in particular ones that regulate cellular cAMP concentration or that are uniquely expressed, may be novel and innovative targets for PAH based on their regulation of muscle tone and proliferation in PAH-PASMCs.

### EXAMPLES

### Materials and Methods

### PASMC Cell Culture

PASMCs (Control, IPAH, and SPAH) were isolated as previously described by Murray et al 2011 and grown in LIFELINE CELL TECHNOLOGY media (containing L-glutamine, recombinant human (rh) Insulin, rh FGF-b, Ascorbic Acid, rh EGF, 1× penicillin and streptomycin, and 10% heat-inactivated fetal bovine serum [FBS]) in a humidified 37° C./5% CO₂ incubator. Cell number and viability was determined using 0.4% Trypan blue (Invitrogen; Carlsbad, Calif.), and a Bright-Line Hemacytometer (Reichart; Depew, N.Y.). PASMC were used in experiments when they reached 70% confluency.

### Human Embryonic Kidney Cell Culture

Human embryonic kidney (HEK) 293 cells were cultured in CORNING CELLGRO Dulbecco's Modification of Eagle's Medium (DMEM) 1× (containing 4.5 g/L glucose, L-glutamine, and sodium pyruvate) with added 10% heat-inactivated FBS and 1× penicillin and streptomycin in a humidified 37° C./5% CO₂ incubator. Cell number and viability was determined using 0.4% Trypan blue (Invitrogen, and a Bright-Line Hemacytometer (Reichart). HEK 293 cells were used in experiments when they reached 70% confluency.

### Animal Models of PAH

Chronic Hypoxic Mouse Model: C57/BL6 mice (3 months old, male) were placed in a hypobaric chamber (0.5 atm) for 4 weeks. Right ventricular hypertrophy was assessed to determine the development of PAH. It was evaluated as the ratio of the weight of the right ventricle to that of the left ventricle plus the septum (Fulton Index). Control mice had an average value of 0.24 and CH mice had an average value of 0.32. This model is commonly used and approved as a valid model of PAH.^{42,43} All animals were cared for in compliance with the guiding principles and approved by the UCSD Institutional Animal Care and Use Committee.

### Chronic Hypoxic Rat Model

Adult Sprague-Dawley rats (250-300 g, male) were placed in a hypobaric chamber (0.5 atm) for 2-4 weeks. Right ventricular hypertrophy was assessed to determine the development of PAH. It was evaluated as the ratio of the weight of the right ventricle to that of the left ventricle plus the septum (Fulton Index). Control rats had an average value of 0.22 and CH rats had an average value of 0.32. This model is commonly used and approved as a valid model of PAH.^{42,43} All animals were cared for in compliance with the guiding principles and approved by the UCSD Institutional Animal Care and Use Committee.

### Monocrotaline (MCT)-Treated Rat Model

Adult Sprague-Dawley rats (250-300 g, male) were injected once intraperitoneally with MCT (60 mg/kg) and sacrificed 2 weeks later. Right ventricular hypertrophy was assessed to determine the development of PAH. It was evaluated as the ratio of the weight of the right ventricle to that of the left ventricle plus the septum (Fulton Index). Control rats had an average value of 0.20 and MCT-treated rats had an average value of 0.34. This model is commonly used and approved as a valid model of PAH.^{42,43} All animals were cared for in compliance with the guiding principles and approved by the UCSD Institutional Animal Care and Use Committee.

### Transfection of PASMCs

PASMCs (0.5-1×10⁶) were transfected with 10 μg of plasmid DNA (e.g., GPR75 Plasmid Vector [using pCMV6-Entry Vector] or Empty Vector Control) using an AMAXA NUCLEOFECTOR device and Program A-033 (AMAXA, Köln, Germany). The cells were then plated into a 6-well plate and incubated in a humidified 37° C./5% CO₂ incubator for 24-72 hrs.

### Transfection of HEK 293 Cells

Prior to transfection (24 hrs) of HEK 293 cells, the cells were first plated into 6-well plates at 50-70% confluency in DMEM with 10% heat-inactivated FBS (no antibiotics). MIRUS TransIT-LT1 Transfection Reagent (7.5 ul) was combined with 250 μL of Opti-MEM I Reduced-Serum Medium and 2.5 μg plasmid DNA (e.g., GPR75 Plasmid Vector [using pCMV6-Entry Vector] or Empty Vector Control). The solution was incubated at room temperature for 30 min; the TransIT-LT1 Reagent: DNA complexes were then added drop-wise. Cells were incubated in a humidified 37° C./5% CO₂ incubator for 24-72 hrs.

### Restriction Digest

2 μL of New England Biolabs Inc. (NEB) Buffer 3 was added with 0.2 μL of Bovine Serum Albumin (BSA, 10 mg/mL), 2 μL of plasmid DNA (0.5 μg/μL-1.5 μg/μL), 1 μL of New England Biolabs Inc. Bgl II (10.00 U/mL) restriction enzyme, and 13.8 μL H₂O. The digest was incubated at 37° C. for 2 hrs and then visualized using ˜500 ng of digest on agarose gel electrophoresis to confirm the size of the insert.

### Real-Time PCR Primer Designs

Primers for each GPCR were designed using the NCBI Entrez search engine and the Primer3 online primer-designing program (from MIT, Cambridge, Mass.) using standard settings. Multiple primer pairs were chosen for each GPCR (ValueGene, San Diego, Calif.) and stored at a concentration of 200 μM.

### Real-Time PCR Protocol

mRNA was extracted from 1×10⁶ Control (Ctrl)-, IPAH-, and SPAH-PASMC and/or from isolated mouse/rat lungs using RNeasy (Qiagen) according to the manufacturer's instructions. cDNA was synthesized using Superscipt III Reverse Transcriptase kit (Invitrogen), as per the manufacturer's instructions. Real-time PCR was performed using 8 ng cDNA, 0.5 μM forward and reverse primers, and qPCR Mastermix Plus for Sybr Green I (Eurogentec, San Diego, Calif.) and an Opticon 2 RT-PCR machine (MJ Research, Waltham, Mass.). The RT-PCR program and RT-PCR primers are shown in Tables 1 and 2, respectively. Primer efficiency was calculated for each primer set before use. Samples were compared using the relative cycle threshold (Cₜ) method, normalizing to 28S or 18S rRNA.

### TAQMAN GPCR Array

GPCR expression was determined using a TAQMAN GPCR array (Life Technologies), according to manufacturer's instructions, with cDNA pooled from Control (Ctrl)- (n=3), IPAH- (n=3), and SPAH-PASMCs (n=3) and the TAQMAN Universal PCR Master Mix. GPCR expression was normalized to that of 18S rRNA.

TABLE 1
Real-time PCR protocol

| Temperature (° C.) | Time | |
|---|---|---|
| 50 | 2 | min |
| 95 | 10 | min |
| 95 | 15 | sec |
| 60 | 30 | sec |
| 72 | 1 | min |

Plate read.
Step 3 and beyond are repeated 34 more times.
Melting curves for samples are constructed by heating the plate from 60° C. to 95° C., and reading the plate every 0.2° C., holding the temperature for 1 sec.

TABLE 2
RT-PCR primers.

| | | SEQ | | SEQ |
|---|---|---|---|---|
| | Forward primer | ID | Reverse primer | ID |
| Gene | (5′ → 3′) | NO: | (5′ → 3′) | NO: |
| ADORA2B | ACCCAGAGGACAGC | 3 | CAGAGCTCCATCTT | 4 |
| | AATGAC | | CAGCCT | |
| BDKRB2 | TCCAGGGAGAGAAC | 5 | AGTACCAGGGAGCG | 6 |
| | ATTTGG | | ACTTGA | |
| CALCRL | CGATATGCACAATT | 7 | TTCCTTAAGAGCTG | 8 |
| | GTCTCCA | | GACTGG | |
| GPR113 | TCTCCAACATGTCC | 9 | ATGGAAGTCGAGCC | 10 |
| | CATCAC | | ACATCT | |
| GPR124 | CTCAGGTCCAGCTT | 11 | CCTTCTGCCTAACG | 12 |
| | CTCCAG | | GCAC | |
| GPR176 | GCAGGCCAGGTTTT | 13 | TGTACCGCCAGTTC | 14 |
| | TAATGA | | ACCAC | |
| GPR75 | GACCTGTACTTTTC | 15 | GCTGTCACTCCACA | 16 |
| | TACTGGCG | | AATGAAG | |
| GPRC5B | ATGTGCTGGCGTTT | 17 | AGGCCAGCTGGAGC | 18 |
| | TCAGAG | | GTC | |
| LPAR1 | CCAGGAGTCCAGCA | 19 | TGTCTCGGCATAGT | 20 |
| | GATGAT | | TCTGGA | |
| mGPR113 | AAGAGGCTCTGTGG | 21 | TACCACCTTGGCCA | 22 |
| | GACTGA | | GTAAGG | |
| mGPR75 | AGGAGCAAGATGCA | 23 | CACCTTCGTGCTGT | 24 |
| | GGAAAA | | TCTTCA | |
| OXTR | CACGAGTTCGTGGA | 25 | TTCTTCGTGCAGAT | 26 |
| | AGAGGT | | GTGGAG | |
| PAR1 | GGGGATCTAAGGTG | 27 | CCGCCTGCTTCAGT | 28 |
| | GCATTT | | CTGT | |
| rGPR113 | TGGTCAGATGAAGG | 29 | CCGGTAAATAACCA | 30 |
| | GTGTGA | | GGCAGA | |
| rGPR75 | GCGGTCAACCTCTC | 31 | GCACCAGAGCACTT | 32 |
| | TACTGC | | TCCTTC | |
| S1P2 | GCAACAGAGGATGA | 33 | GGAGTACCTGAACC | 34 |
| | CGATGA | | CCAACA | |
| VIPR1 | GCTGCACCTCGATC | 35 | CTGGCTATGCGTGC | 36 |
| | ATCTG | | TGG | |
| 18S | GTAACCCGTTGAAC | 37 | CCATCCAATCGGTA | 38 |
| | CCCATT | | GTAGCG | |
| 28S | GCCTAGCAGCCGAC | 39 | AAATCACATCGCGT | 40 |
| | TTAGAA | | CAACAC | |

All primers are for human genes unless otherwise stated (m = Mouse, r = Rat, Tₘ = 60° C.).

cAMP Radioimmunoassay

Control-PASMCs, PAH-PASMCs or HEK 293 cells were seeded at 30,000 cells/well in a 24-well plate. After 24 hrs the cells were serum starved for 2 hrs and incubated in the absence or presence of 3-isobutyl-1-methylxanthine (IBMX, 200 μM), a competitive non-selective PDE inhibitor, for 30 min at 37° C. GPCR agonists of interest were then added to the cells with/without forskolin (FSK, 1 μM, a direct activator of AC, which enhances Gαₛ-GPCR promoted cAMP formation and helps demonstrate Gαᵢ-GPCR activation) for 10 min at 37° C. 10 uM FSK (alone) was used as a positive control. After the incubations, the media was aspirated and 150 μL of 7.5% trichloroacetic acid (TCA) was added to each well.

In studies of conditioned media from hybridomas, control- and IPAH-PASMCs were seeded at 30,000 cells/well in a 24-well plate (˜30,000 cells/well) in Vasculife Smooth Muscle cell media (Life Technologies) and cultured at 37° C., in a humidified 5% CO2 incubator. After 24 hr, the cells were serum-starved for 4 hr, antibody (1 μg/mL ascites, 1 μg/mL mouse IgG or (1:100 conditioned media from hybridoma cultures) was added for 2 hrs, and then 200 μM IBMX was added 30 min prior to addition of 10 μM forskolin for 10 min and then samples were assayed for cAMP.

Assay tubes were filled with 1 mL of 10 mM sodium acetate buffer (pH 4.75) and a standard curve was constructed by serial dilution of stock 5 μM cAMP (Millipore, Billerica, Mass.). An appropriate amount of sample was added to the assay tubes and the samples were acetylated by addition of 20 μL triethylamine (Sigma) and 10 μl acetic anhydride (Sigma). 100 μL of the sodium acetate buffer was added to each well of a 96-well filter plate to prepare the plate and then was removed by vacuum. 50 μL of diluted, acetylated sample was added to each well along with 25 μL diluted antibody [1:1000, 6 μL primary cAMP antibody (Millipore) in 6 mL γ-globulin buffer (100 mg human γ-globulin/100 mL 50 mM NaAc pH 4.75)] and 25 μL diluted ¹²⁵I radioactivity [16 acetylated adenosine 3′,5′-cyclic phosphoric acid, 2′-O-succinyl [¹²⁵I]-iodotyrosine methyl ester (PerkinElmer) in 3 mL γ-globulin buffer so that 0.001 mCi of ¹²⁵I could be added to each sample. The 96-well filter plate containing the sample was incubated overnight at 4° C.

Following overnight incubation, 50 μL secondary antibody (BIOMAG Goat anti-Rabbit IgG 8-4300D, Qiagen) was added to each well and the plate was incubated for 1 hr at 4° C. The wells were then washed with 100 μL of 12% polyethylene glycol in 10 mM sodium acetate (pH 6.2) three times. The base of the plate was then removed with a MultiScreen Punch Kit (Millipore) into fresh assay tubes and then counted on the WIZARD2 Automatic Gamma Counter (PerkinElmer).

### [³H]Thymidine Incorporation Assay

Ctrl- and PAH-PASMCs or HEK 293 cells were seeded at 30,000 cells/well on a 6-well plate (Greiner Bio-One, Monroe, N.C.) for 24 hr and then serum-starved for a further 24 hrs. GPCR agonists/antagonists/GPR75 antibody in the absence or presence of 200 μM IBMX and [³H] thymidine (1 μCi/ml) were added to the cells for 24-48 hr. Following incubation, cells were washed with a large volume of cold phosphate buffer saline (PBS) and then twice with a large volume of cold 7.5% TCA. The precipitated material was dissolved with 0.5 M NaOH and combined with 3 mL of scintillation fluid (Ecoscint O, National Diagnostics) and radioactivity was determined using a liquid scintillation counter (Beckman Coulter LS 1801).

### Protein Analysis

Cells were washed with cold PBS on ice and lysed in 80 μL-150 μL of lysis buffer (Novagen Cytobuster protein extraction reagent). Protein concentration was determined using a Bio-Rad Protein Assay Dye Reagent according to the manufacturer's instructions.

Lungs and hearts were isolated from control and PAH animals and 10 mg of tissue was homogenized using a glass homogenizer in 400 μL of 1× lysis buffer (Cell Biolabs, Inc.). Samples were then centrifuged at 1200 rpm for 10 min at 4° C. and the supernatant was collected. Protein concentration was determined using a Bio-Rad Protein Assay Dye Reagent Concentrate, according to the manufacturer's instructions.

### Western Blots

7.5 μl of NUPAGE LDS Sample Buffer 4× (Invitrogen), 0.75 μl of reducing agent (2-mercaptoethanol for electrophoresis >=98%, Sigma), and an appropriate amount of lysis buffer were added to purified protein samples (to give a final protein concentration of 1-5 μg protein/20 mL). Samples were loaded onto pre-cast 4-12% gel (Invitrogen) and run for 1 hr at 200V, 40 mA, and 25W. Gels were incubated in 10% methanol transfer buffer for 10 min and protein was transferred to polyvinylidene fluoride (PVDF) membrane using the iBlot (Invitrogen). Membranes were then blocked in 5% milk or BSA (in PBS Tween [PBST], dependent upon antibody) for 1 hr. Primary antibody [1:1000 dilution in 1% milk/BSA] was then added and the membrane was incubated overnight. The following day, it was washed 3 times (10 min/wash) with PBST at RT. Secondary anti-rabbit or anti-mouse antibody (1:5000 or 1:3000 respectively in 1% milk/BSA, AbCam) was added and incubated for 1 hr. The membranes were washed 3 additional times (10 min/wash) with PBST at RT and ECL luminescence (GE Healthcare) was added for detection.

### Immunofluorescence

Ctrl- and PAH-PASMCs were grown on sterile 12 mm coverslips pre-coated with poly-D-lysine. Cells were washed twice with PBS at 37° C., then 1 mL of fixative (2% paraformaldehyde) was added for 10 min. Aldehyde groups were then quenched by incubation with 100 mM glycine (in PBS, pH 7.4) for 10 min. Cells were washed twice with PBS and then with blocking buffer (1% BSA/PBS/0.05% Tween) for 30 min at room temperature and then with primary antibody in that buffer and incubated (1:250) overnight at 4° C. Cells were then washed 3 times with wash medium (PBS/0.1% Tween20) for 5 min each at room temperature. Blocking buffer was then added containing secondary antibodies (1:250) for 60 min at room temperature. Cells were washed 6 times for 5 min each in wash medium at room temperature and then incubated with 4′,6-diamidino-2-phenylindole (DAPI, 1:5000) for 20 min in the dark and, mounted on slides with 10 μL of gelvatol. Slides were left to dry overnight at 4° C. Images were acquired using a confocal microscope.

Lungs from Control and Chronic Hypoxic rats were cut into semi-thin sections (10 microns). Sections were fixed in cold acetone, blocked with 4% BSA in 0.1% Tween and PBS, and incubated with primary antibodies (1:100) in 4% BSA in 0.1% Tween and PBS, and incubated with Alexa-conjugated secondary antibody (1:250). Samples were mounted in gelvatol. Specificity of staining was determined by omission of the primary antibody. Images were obtained using a laser scanning confocal microscope.

### Deglycosylation Assay

1-20 μg of lysate was combined with 1 μL of 10× Glycoprotein Denaturing Buffer (NEW ENGLAND BIOLABS Inc.) and H₂O in 10 μL total volume. The sample was denatured by heating at 100° C. for 10 min. The reaction volume was increased to 20 μL by adding 2 μL of 10×G7 Reaction Buffer, 2 μL of 10% NP-40, H₂O and 1-2 μL PNGase F (500,000 U/mL, NEW ENGLAND BIOLABS Inc.) and then incubated at 37° C. for 1 hr.

### Microparticle Isolation Protocol

Platelet-poor plasma (PPP) was prepared from patients. Platelets were removed from plasma by centrifugal force: first at 1500×g for 25 min, then by a second centrifugation at 15,000×g for 45 min. The PPP (supernatant) was removed, placed into ultracentrifuge tubes and topped off with phosphate buffer saline (PBS) and then centrifuged at 100,000×g for 75 min at room temperature. All but 100 μl of supernatant/pellet at the bottom of the tube (microparticle-enriched fraction) was removed for protein and western blot analysis.

### Preparation of Hybridoma Media

Hybridoma clones were grown in DMEM/F12 media, supplemented with L-glutamine, HEPES, 10% heat inactivated Fetal Bovine Serum (FBS) and penicillin/streptomycin. Each hybridoma line was expanded in a 225 cm² vented cap flask in a volume of 50 mL. Hybridoma cells were grown for ˜2 weeks. Cells were removed from media via centrifugation once at 233×g and then at 931×g). Supernatants were used at a 1:100 dilution in cAMP accumulation assays.

### Statistical Analysis

Values are expressed as mean±SEM. Statistical significance was determined via an unpaired or paired Student's test or an Anova when applicable. A value of P<0.05 was considered statistically significant.

### Results Part I

### Quantification of GPCR Expression in Ctrl-, IPAH-, and SPAH-PASMCs

Using an unbiased approach, a TAQMAN GPCR array, GPCR expression in Ctrl-, IPAH-, and SPAH-PASMCs was identified. This approach was used to identify GPCRs that are higher or uniquely expressed in patients with PAH among the 384 genes that were analyzed (29 housekeeping genes+355 non-chemosensory GPCRs). Results showed that that Ctrl-PASMCs (n=3) express 135 GPCRs (including 56 orphan receptors), IPAH-PASMCs (n=3) express 115 GPCRs (51 orphan receptors) and SPAH-PASMC (n=3) express 81 GPCRs (32 orphan receptors) (Table 3). The non-orphan expressed GPCRs in each cell type were classified further and separated according to their linkage to specific G proteins (Gαₛ/Gαᵢ/Gα_{q/11}/Gα_{12/13}, Table 4) by using the 2011 BJP (British Journal of Pharmacology) Guide to Receptors and Channels and the IUPHAR Database of Receptors and Ion Channels as references. The results for the three highest expressed Gαₛ-, Gαᵢ-, Gα_{12/13}-linked GPCRs from Ctrl-, IPAH-, and SPAH-PASMCs are shown in Tables 5-8. The values are shown as ΔCₜ, whereby the cycle threshold (CO for each GPCR RNA is normalized to that of 18S rRNA; a lower Cₜ value thus indicates higher expression. Categorizing the receptor expression in each group allowed for the determination of the expression of GPCRs that regulate cAMP and highlighted potential therapeutic targets, since increases in cAMP accumulation decrease proliferation and vasodilate PASMC.^{18-20,26-31}

TABLE 3
GPCR expression in Ctrl-PASMC (n = 3), IPAH-PASMC (n = 3), and
SPAH-PASMC (n = 3)

| | | | | Expressed | Expressed |
|---|---|---|---|---|---|
| | Total | Undetectable | Expressed | orphan | non-orphan |
| Cell Type | GPCRs | GPCRs | GPCRs | GPCRs | GPCRs |
| Ctrl- | 355 | 220 | 135 | 56 | 79 |
| PASMC | | | | | |
| IPAH- | 355 | 240 | 115 | 51 | 64 |
| PASMC | | | | | |
| SPAH- | 355 | 274 | 81 | 32 | 49 |
| PASMC | | | | | |

TABLE 4
GPCRs in Ctrl-PASMC (n = 3), IPAH-PASMC (n = 3), and
SPAH-PASMC (n = 3) classified by their G protein-coupling.
(Many GPCRs have multiple coupling so may appear in more than
1 category)
GPCRs-non-orphans

| | | | | Gα_{12/13}- |
|---|---|---|---|---|
| CellType | Gαₛ-coupled | Gαᵢ-coupled | Gα_{q/11}-coupled | coupled |
| Ctrl-PASMC | 23 | 40 | 36 | 9 |
| IPAH-PASMC | 19 | 34 | 31 | 8 |
| SPAH-PASMC | 13 | 22 | 20 | 7 |

TABLE 5
Three highest expressed Gαₛ-linked GPCRs in
Ctrl-PASMC (n = 3), IPAH-PASMC (n = 3), and SPAH-PASMC (n = 3).
ΔCₜ values averaged and normalized with 18S (lower values represent
higher expression)

| | | IPAH-PASMC | | SPAH-PASMC | |
|---|---|---|---|---|---|
| Ctrl-PASMC | | Gαₛ-coupled | | Gαₛ-coupled | |
| Gαₛ-coupled GPCR | ΔCₜ | GPCR | ΔCₜ | GPCR | ΔCₜ |
| ADORA2B | 18.7 | ADRB2 | 15.0 | VIPR1 | 15.0 |
| VIPR1 | 19.2 | P2RY11 | 16.0 | ADRB2 | 18.0 |
| CALCRL | 20.2 | PTGIR | 17.0 | PTGIR | 18.0 |

TABLE 6
Three highest expressed Gαᵢ-linked GPCRs in Ctrl-PASMC (n = 3),
IPAH-PASMC (n = 3), and SPAH-PASMC (n = 3). ΔCₜ values
averaged and normalized with 18S (lower value represents higher
expression)

| | | IPAH-PASMC | | SPAH-PASMC | |
|---|---|---|---|---|---|
| Ctrl-PASMC | | Gαᵢ-coupled | | Gαᵢ-coupled | |
| Gαᵢ-coupled GPCR | ΔCₜ | GPCR | ΔCₜ | GPCR | ΔCₜ |
| LPAR1 | 15.2 | LPAR1 | 14.1 | SSTR1 | 16.0 |
| OXTR | 16.1 | CHRM2 | 15.0 | LPAR1 | 16.1 |
| PAR1 | 17.2 | SSTR1 | 15.1 | GABBR1 | 18.0 |

TABLE 7
Three highest expressed Gα_{q/11}-linked GPCRs Ctrl-PASMC (n = 3),
IPAH-PASMC (n = 3), and SPAH-PASMC (n = 3). ΔCₜ values averaged
and normalized with 18S (lower value represents higher expression)

| Ctrl-PASMC | | IPAH-PASMC | | SPAH-PASMC | |
|---|---|---|---|---|---|
| Gα_{q/11}-coupled | | Gα_{q/11}-coupled | | Gα_{q/11}-coupled | |
| GPCR | ΔCₜ | GPCR | ΔCₜ | GPCR | ΔCₜ |
| OXTR | 16.1 | PAR1 | 10.0 | PAR1 | 14.1 |
| BDKRB2 | 16.2 | BDKRB2 | 13.0 | OXTR | 14.1 |
| PAR1 | 17.2 | BDKRB1 | 14.0 | LPAR1 | 16.1 |

TABLE 8
Three highest expressed Gα_{12/13}-linked GPCRs Ctrl-PASMC (n = 3),
IPAH-PASMC (n = 3), and SPAH-PASMC (n = 3). ΔCₜ values averaged
and normalized with 18S (lower value represents higher expression)

| | | | | SPAH-PASMC | |
|---|---|---|---|---|---|
| Ctrl-PASMC | | IPAH-PASMC | | Gα_{12/13}- | |
| Gα_{12/13}-coupled | | Gα_{12/13}-coupled | | coupled | |
| GPCR | ΔCₜ | GPCR | ΔCₜ | GPCR | ΔCₜ |
| LPAR1 | 15.2 | PAR1 | 10.0 | PAR1 | 14.1 |
| S1PR2 | 17.2 | LPAR1 | 14.1 | LPAR1 | 16.1 |
| PAR1 | 17.2 | S1PR2 | 15.0 | S1PR2 | 16.1 |

### Highest Expressed GPCRs in Ctrl-PASMC Confirmed by Independent Real-Time PCR

The highest expressed GPCRs, determined by the TAQMAN GPCR array, were confirmed by real-time PCR to ensure validity of the array data. The results confirmed the expression of the 3 highest expressed Gαₛ-, Gαᵢ-, and Gα_{q/11}-coupled GPCRs and orphan receptors in Ctrl-PASMCs (Table 9). Data from the individual real-time PCR studies generally correlated well with values from the microarray. The overall r² value was calculated to be 0.70 (FIG. 3A).

TABLE 9
Highest expressed GPCRs in Ctrl-PASMCs confirmed by independent
real-time PCR.

| | |
|---|---|
| Microarray | Real-time PCR |

| Gas-coupled | ΔCt | Gas-coupled | ΔCT |
|---|---|---|---|
| Adenosine A2B | 18 | Adenosine A2B | 17 |
| Receptor | | Receptor | |
| VIP Receptor 1 | 19 | VIP Receptor 1 | 18 |
| Calcitonin Receptor- | 20 | Calcitonin | 20 |
| Like | | Receptor-Like | |
| Gai-coupled | 15 | Gai-coupled | 15 |
| LPAR1 | 16 | LPAR1 | 16 |
| Oxytocin Receptor | 17 | Oxytocin Receptor | 16 |
| PAR1 | | PAR1 | |
| Gaq-coupled | | Gaq-coupled | |
| Oxytocin Receptor | 16 | Oxytocin Receptor | 16 |
| Bradykinin Receptor | 16 | Bradykinin | 18 |
| B2 PAR1 | | Receptor B2 PAR1 | |
| PAR1 | 17 | PAR1 | 16 |
| Orphan Receptor | | Orphan Receptor | |
| GPRC5B | 15 | GPRC5B | 16 |
| GPR124 | 15 | GPR124 | 14 |
| GPR176 | 15 | GPR176 | 15 |

Lower ΔCt = Higher Expression

### Results Part II

### Validation of the GPCR Array

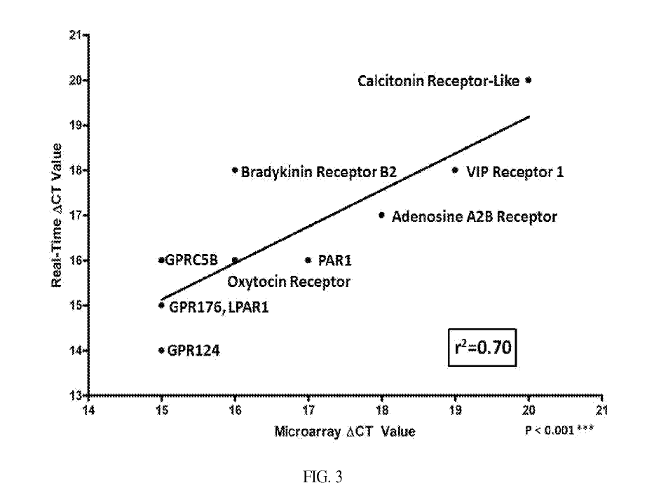

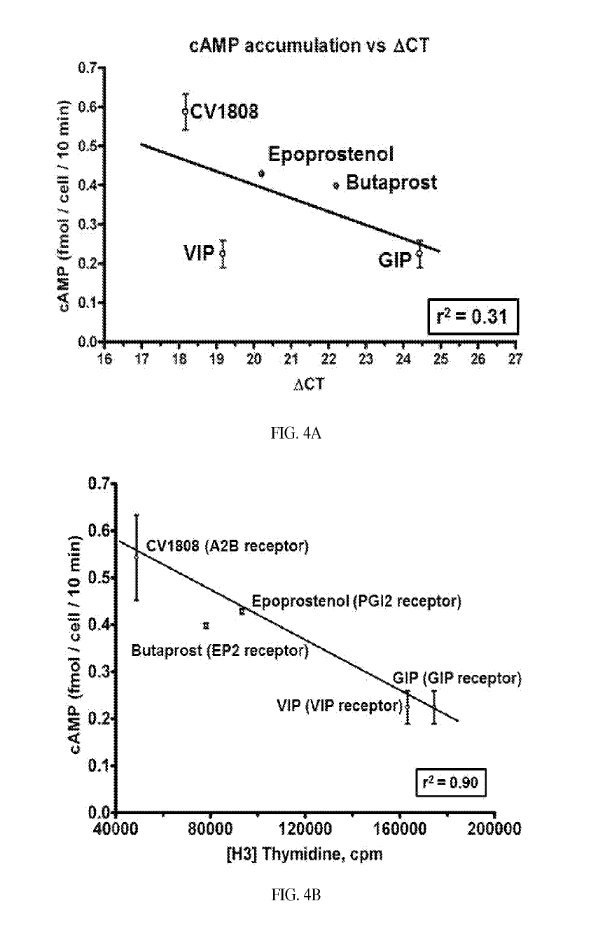

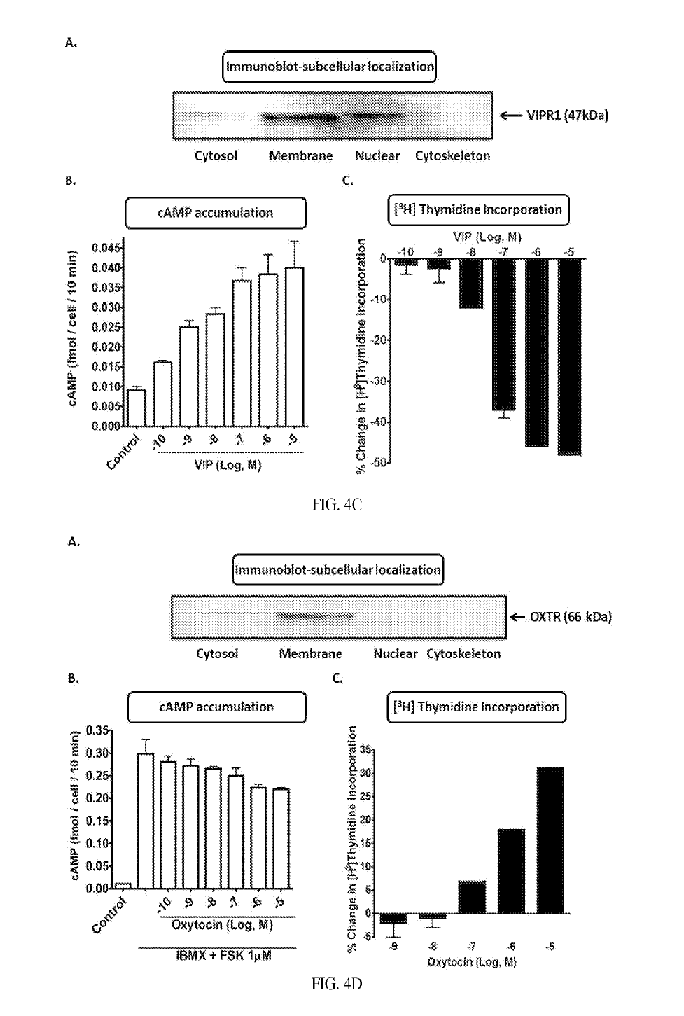

To further validate the TAQMAN GPCR array, functional studies on the Gαₛ-coupled GPCRs that were expressed at high, intermediate and low levels in Ctrl-PASMCs were performed in order to determine if agonist-induced cAMP accumulation of these GPCRs correlated with their mRNA expression. The highest, intermediate and lowest Gαₛ-coupled GPCRs tested were the adenosine 2B receptor (A2BR, ΔCₜ=18), the vasoactive intestinal peptide receptor (VIPR1, ΔCₜ=19), the prostacyclin receptor (IPR, ΔCₜ=20), the prostaglandin E2 receptor (EP2R, ΔCₜ=22) and the gastric inhibitory polypeptide receptor (IPR, ΔCₜ=20). cAMP accumulation (fmol cAMP/cell/10 min treatment with agonist) in response to receptor agonists, CV1808 (A2BR, 1 μM:0.6 fmol), VIP (VIPR1, 1 μM: 0.2 fmol), epoprostenol (IPR, 10 μM: 0.4 fmol), butaprost (EP2R, 1 μM: 0.4 fmol) and GIP (GIPR, 1 μM: 0.2 fmol) correlated with receptor mRNA expression (r²=0.31, FIG. 4A). The concentrations of agonists used were previously shown to produce a maximal response under the conditions tested.

The ability of the agonists was tested and the results are shown (in FIG. 4A) to decrease proliferation of PASMCs (assayed by [³H] thymidine incorporation, which measures DNA synthesis) and found that agonist-induced cAMP accumulation correlated with anti-proliferative effect (FIG. 4B).

Also evaluated were cAMP formation and anti-proliferative response mediated by the Vasoactive Intestinal Peptide (VIP), the second highest expressed Gαₛ-coupled GPCR in Ctrl-PASMCs (Table 9). As shown in FIG. 4C, the VIPR1 agonist VIP dose-dependently increases cAMP and this increase in cAMP corresponds to a decrease in the proliferation of PASMCs. A lower concentration of VIP increases cAMP accumulation without altering PASMC proliferation; these data suggesting a threshold of cAMP is needed before it affects PASMC proliferation. It was confirmed that VIPR1 is expressed on the membrane of PASMCs by performing immunoblot with different subcellular fractions (FIG. 4C).

Investigations were conducted regarding expression and function of the oxytocin receptor, the second highest expressed Gαᵢ-coupled GPCR in PASMCs (Table 9). It was found that the oxytocin receptor is expressed on the membrane of Ctrl-PASMCs and that its agonist oxytocin decreased cAMP levels and increased proliferation of PASMCs (FIG. 4D). Akin to the findings for VIP, lower concentrations of oxytocin decreased cAMP levels than were able to increase proliferation of PASMCs.

### Results Part III

### Altered Expression of GPCRs in PAH-PASMCs

The altered expression of GPCRs in PAH-PASMCs could provide insight into the possible mechanisms that contribute to the development and progression of the disease as well as defining possible targets for future therapy. Using the data derived from the TAQMAN GPCR array, we sought to identify GPCRs that showed the greatest differences in expression between Ctrl- and PAH-PASMCs or GPCRs that were expressed in one cell type but not another (uniquely expressed GPCRs). GPCR expression was compared between Ctrl- and PAH-PASMCs (both obtained from patients with idiopathic and secondary PAH).

FIG. 5A shows Venn diagrams that depict increases in GPCR expression (>2-fold) and decreases in GPCR expression (<0.5-fold) in IPAH- and SPAH-PASMCs compared to Ctrl-PASMCs. The number in each circle indicates the number of GPCRs that increased/decreased in expression in either IPAH or SPAH while the number in the overlapping area of both circles indicates shared GPCRs that increase/decrease in expression in both forms of PAH. 66 GPCRs were identified as having increased expression in IPAH-PASMCs compared to Ctrl-PASMCs, 16 GPCRs were identified as having increased expression in SPAH-PASMCs compared to Ctrl-PASMCs, and 41 GPCRs were identified as having increased expression in both IPAH and SPAH compared to control cells. IPAH thus has 107 (66+41) GPCRs with increased expression while SPAH has 57 (16+41) GPCRs with increased expression compared to Ctrl-PASMCs.

Tables 10-13 show the 3 GPCRs that have the greatest increase or decrease in expression in IPAH-/SPAH-PASMCs compared to Ctrl-PASMCs. These GPCRs could be novel therapeutic targets due to their altered expression in the diseased cells.

TABLE 10
The 3 GPCRs with the greatest increase in mRNA expression in IPAH
compared to Ctrl-PASMCs.

| | | | Principal |
|---|---|---|---|
| Gene | Full Name | Fold Increase | Transduction |
| ADRA1D | Adrenergic α-1D | 549 | Gα_{q/11} |
| CHRM2 | Cholinergic muscarinic 2 | 290 | Gαᵢ |
| ADRB2 | Adrenergic β-2 | 287 | Gαₛ |

TABLE 11
The 3 GPCRs with the largest decrease in mRNA expression in IPAH
compared to Ctrl-PASMC.

| Gene | Full Name | Fold Decrease | Principal Transduction |
|---|---|---|---|
| OXTR | Oxytocin Receptor | −3.70 | Gα_{q/11}, Gαᵢ |
| LPHN1 | Latrophilin 1 | −3.64 | Class B Orphan |
| GPRC5B | GPCR, family 5CB | −3.61 | Class C Orphan |

TABLE 12
The 3 GPCRs with the greatest increase in mRNA expression in SPAH
compared to Ctrl-PASMC.

| | | | Principal |
|---|---|---|---|
| Gene | Full Name | Fold Increase | Transduction |
| CXCR4 | Chemokine (C—X—C motif) | 69.3 | Gαᵢ |
| | 4 | | |
| SSTR1 | Somatostatin receptor 1 | 35.7 | Gαᵢ |
| CHRM5 | Cholinergic muscarinic 5 | 35.4 | Gα_{q/11} |

TABLE 13
The 3 GPCRs with the largest decrease in mRNA expression in SPAH
compared to Ctrl-PASMCs

| | | | Principal |
|---|---|---|---|
| Gene | Full Name | Fold Decrease | Transduction |
| GPR161 | GPCR 161 | −7.26 | Class A Orphan |
| GPR153 | GPCR 153 | −3.63 | Class A Orphan |
| PTGFR | Prostaglandin f receptor | −3.51 | Gα_{q/11} |

Multiple GPCRs that are uniquely expressed in PAH-PASMCs compared to Ctrl-PASMC: 26 GPCRs were uniquely expressed in either IPAH or SPAH-PASMCs compared to control cells. Of these 26 GPCRs, only 2 were expressed in both IPAH- and SPAH-PASMCs: GPR75 and GPR113, both of which are orphan receptors (Tables 14 and 15). IPAH was associated with a much greater number of uniquely expressed GPCRs than was SPAH.

TABLE 14
Uniquely expressed GPCRs in IPAH-PASMC compared
to control-PASMCs.
Uniquely Expressed GPCRs in IPAH

| | ΔCt |
|---|---|
| Gas-coupled | |
| LPAR3 | 16 |
| ADCYAP1R1 | 19 |
| DRD5 | 20 |
| MC5R | 21 |
| GPR92 | 20 |
| FPRL2 | 21 |
| GALR2 | 21 |
| GPR43 | 21 |
| GRM5 | 21 |
| Gai-coupled | |
| LPAR3 | 16 |
| PTGER3 | 16 |
| P2RY13 | 19 |
| PTAFR | 20 |
| RLN3R1 | 20 |
| GRM4 | 20 |
| GALR2 | 21 |
| Gaq-coupled | |
| LAPR3 | 16 |
| FPR1 | 19 |
| PTAFR | 20 |
| Orphan Receptor | |
| GPR63 | 16 |
| LPHN3 | 19 |
| GPR113 | 19 |
| GPR75 | 19 |
| MRGPRE | 19 |
| GPR45 | 20 |
| GPR35 | 20 |
| BAI3 | 20 |
| CCRL2 | 20 |
| GPR142 | 21 |
| GAPR171 | 21 |

Lower ΔCt = Higher Expression

TABLE 15
Uniquely expressed GPCRs in SPAH-PASMCs compared to
control-PASMC.
Uniquely Expressed GPCRs in SPAH

| | ΔCt |
|---|---|
| Gai-coupled | |
| HTR1D | 20 |
| HTR1B | 21 |
| Gaq-Coupled | |
| P2RY2 | 20 |
| Orphan Receptor | |
| GPR113 | 20 |
| GPR75 | 20 |

Lower ΔCt = Higher Expression

### Results Part IV

Expression of GPR113 in PASMC from PAH Patients and Animals with Experimental PAH

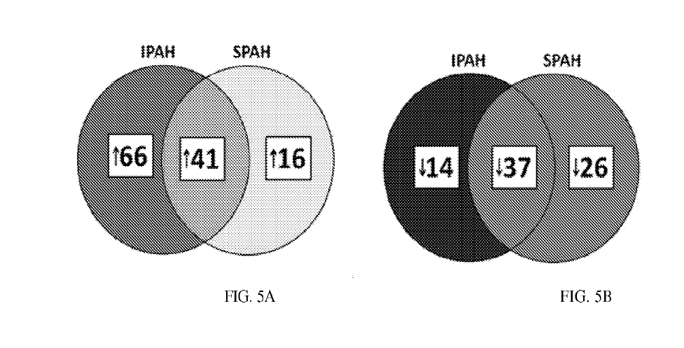

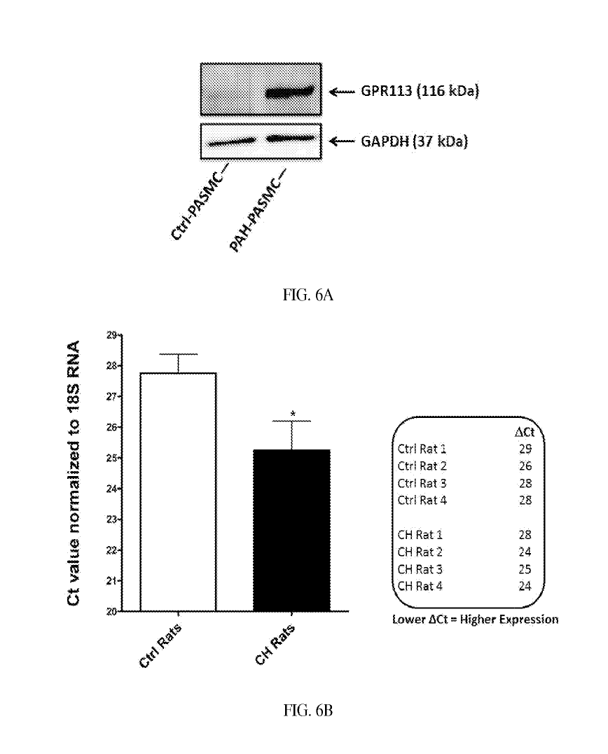

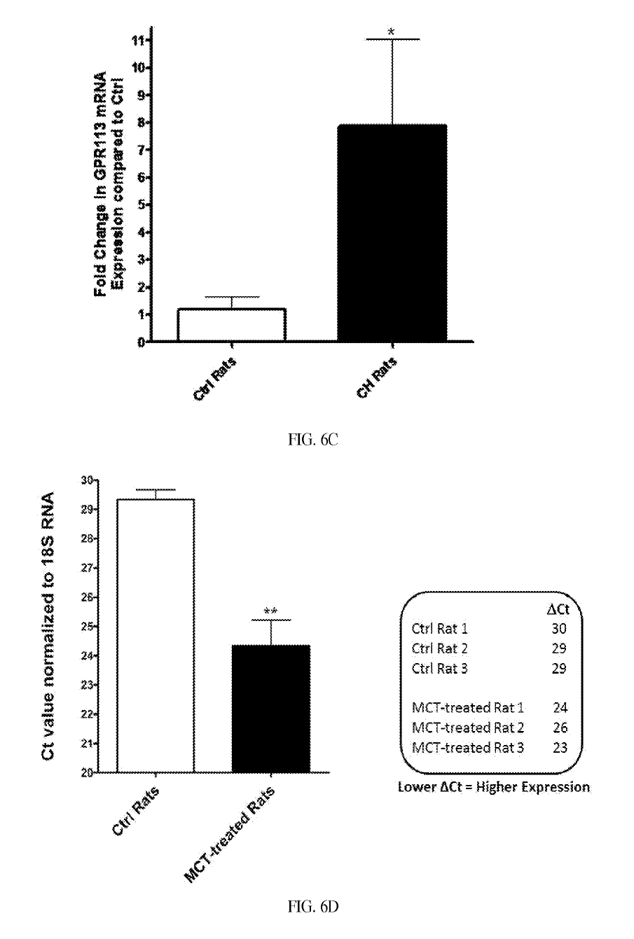

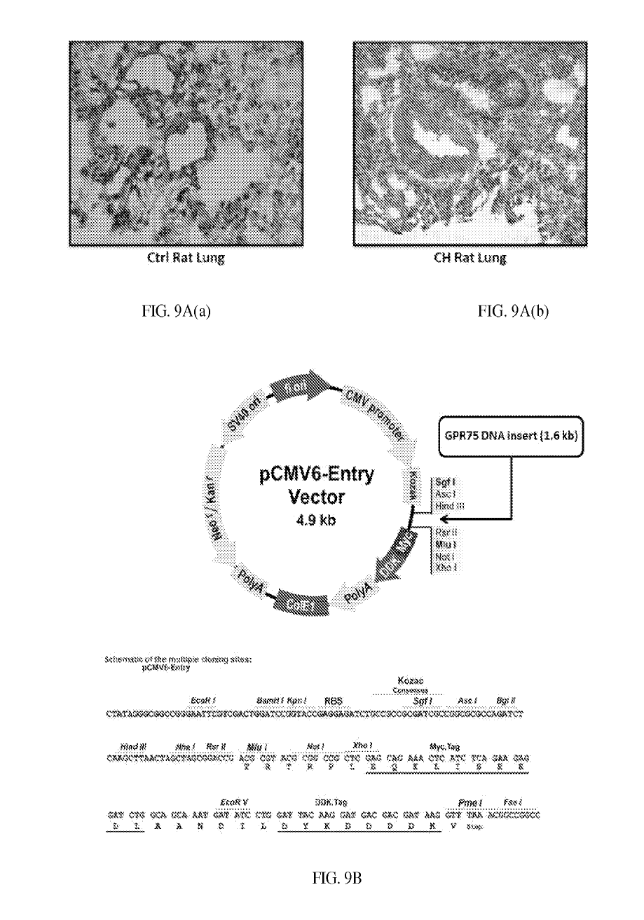

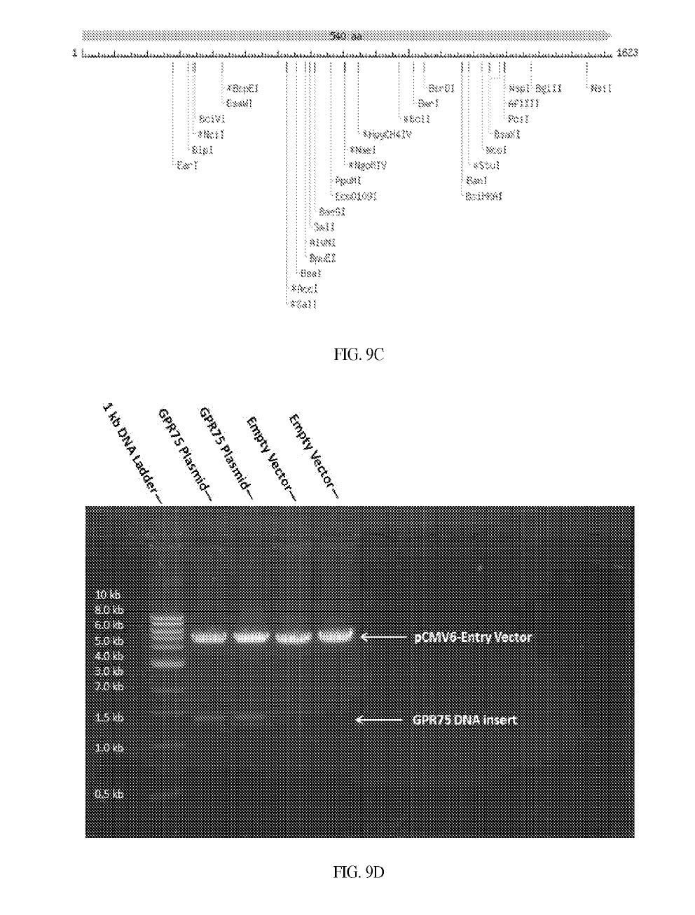

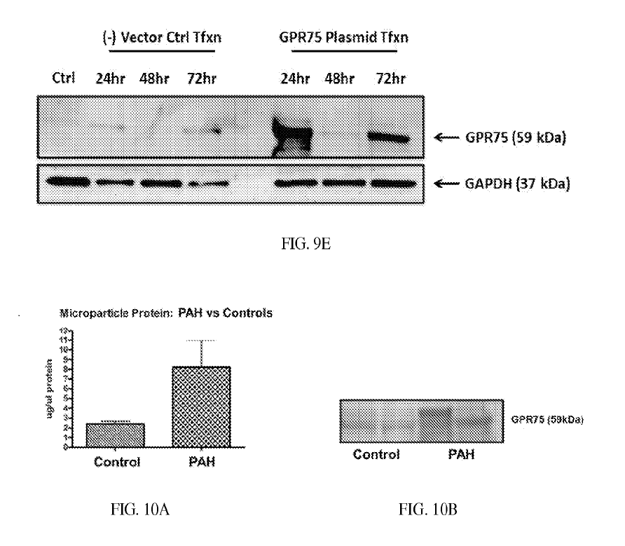

Protein expression of GPR113, one of the orphan GPCRs whose mRNA was uniquely expressed in PAH-PASMCs, was assessed in Ctrl- and PAH-PASMCs cells (FIG. 6A). Each PAH-PASMC (both IPAH and SPAH) sample had at least a 4-fold increase in protein expression of GPR113 compared to control. Results in the chronic hypoxic (CH) and monocrotaline (MCT)-treated rat models of PAH tested whether this expression was specific to PAH in humans. These models are commonly used and accepted as being valid ones of PAH, and show vascular remodeling FIG. 9A).^{42,43} Similar results of increased GPR113 mRNA expression were found in the animal models of PAH compared to controls (FIGS. 6B-E).

### Results Part V

GPR75 Has Increased mRNA and Protein Expression in PAH

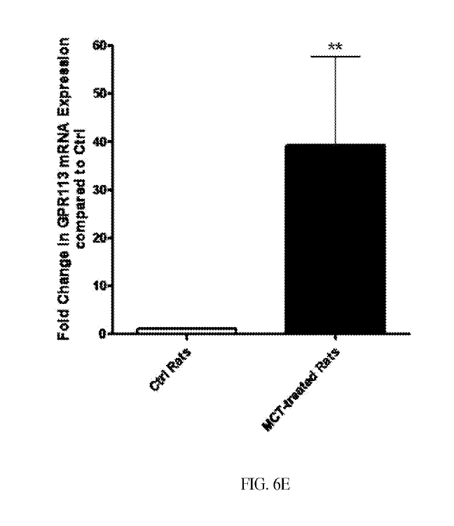

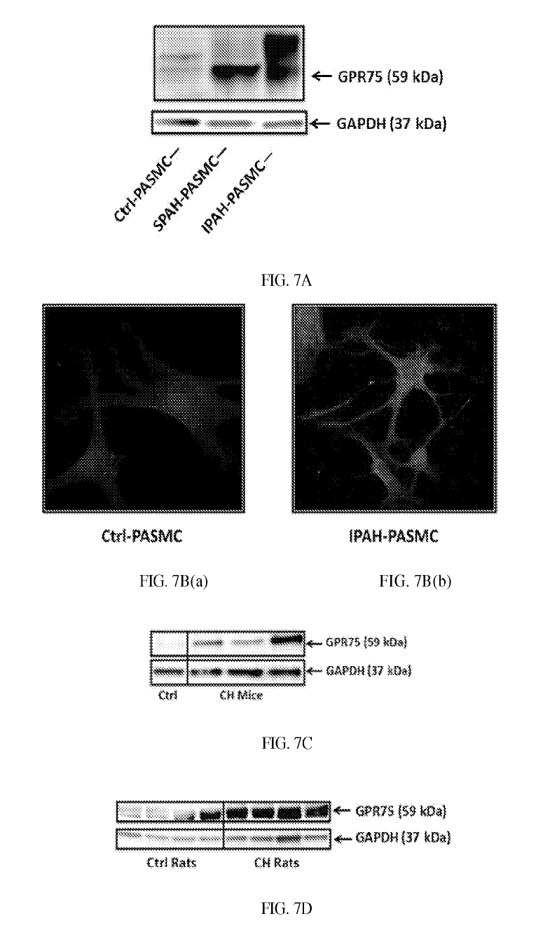

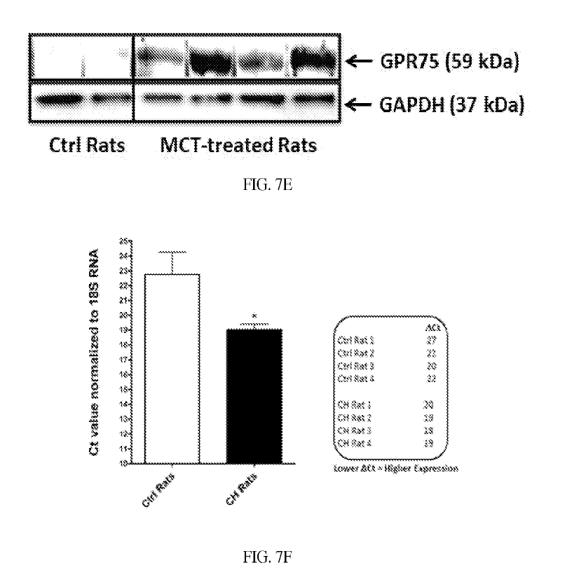

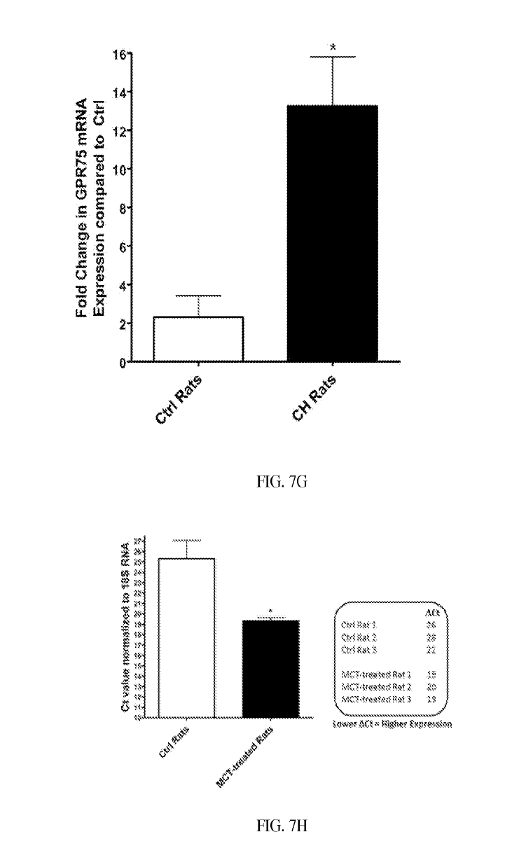

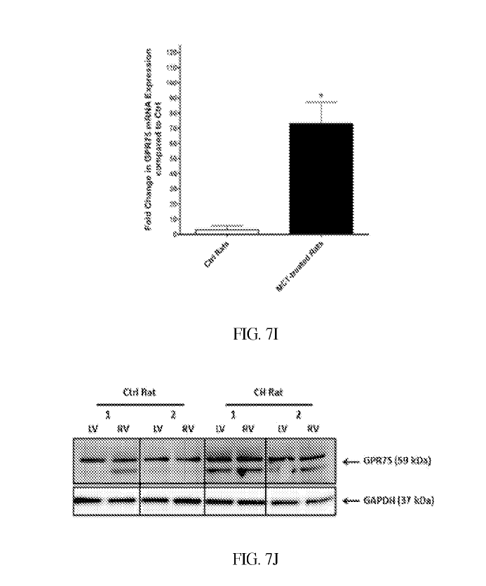

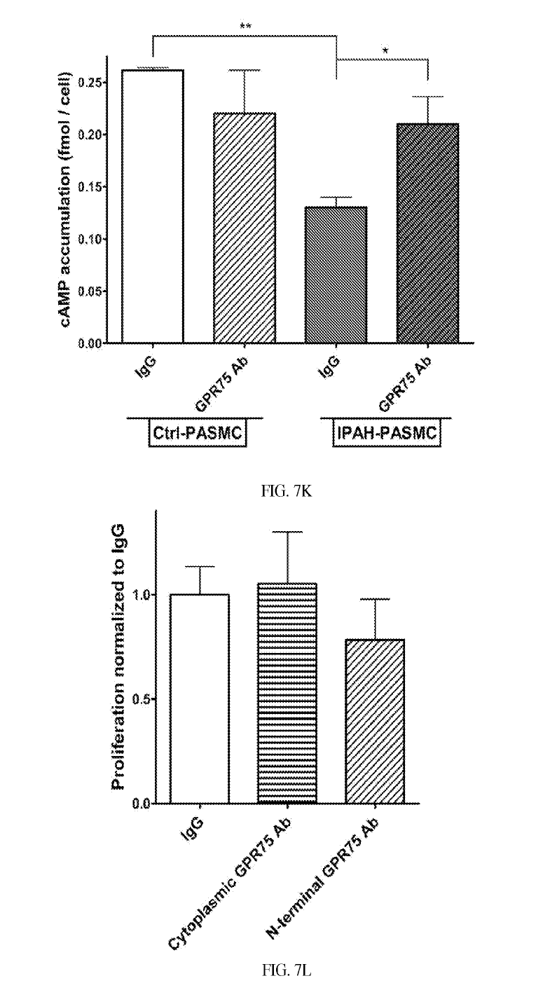

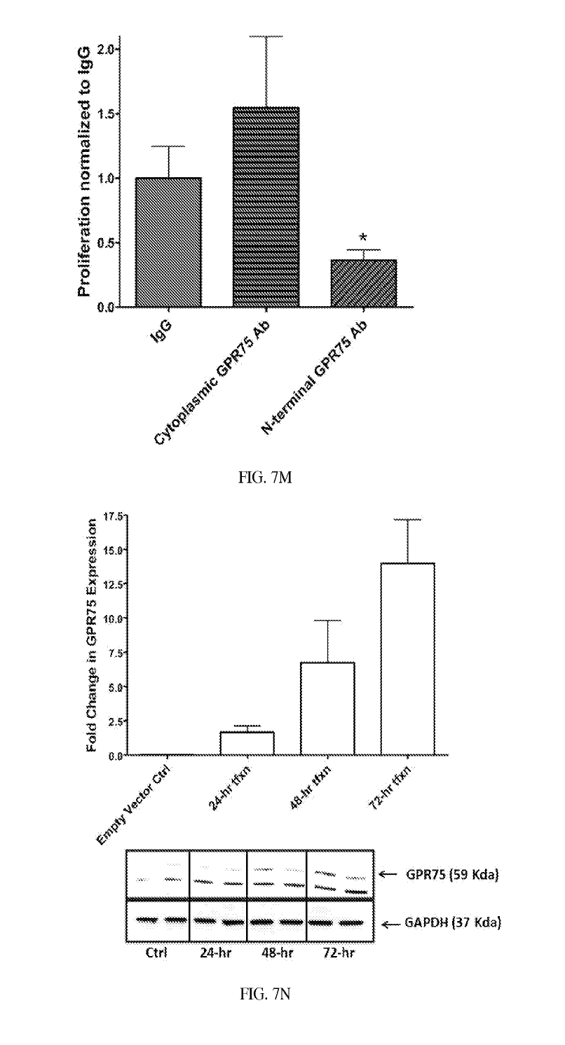

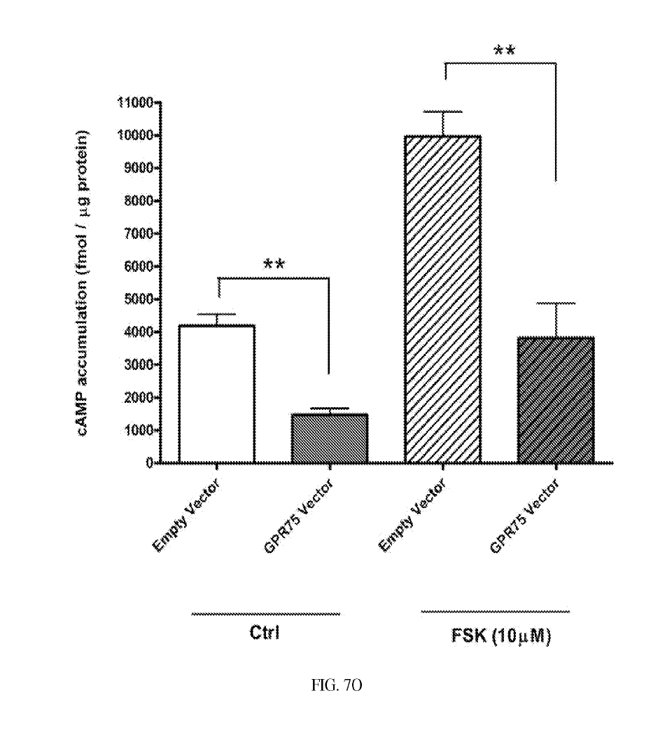

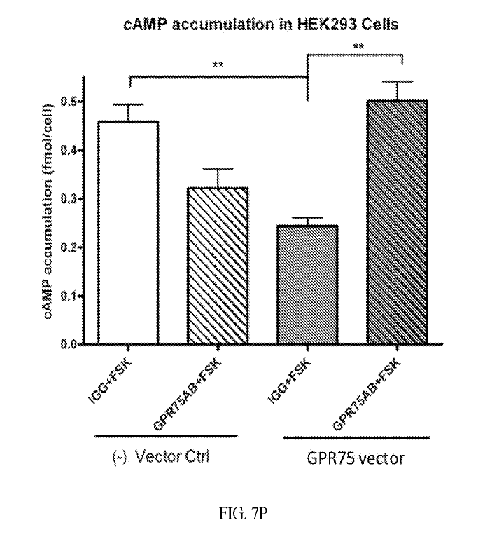

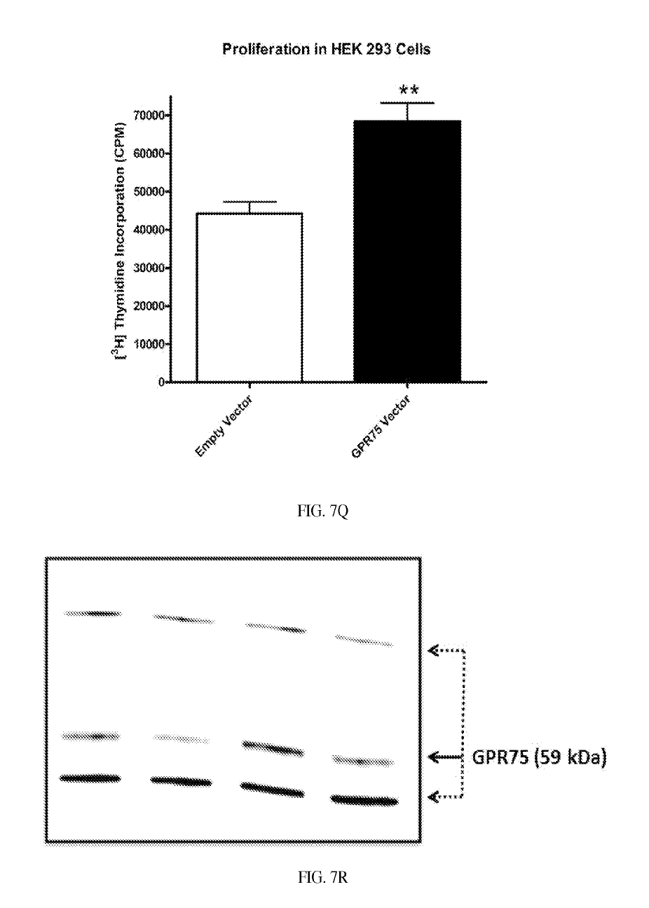

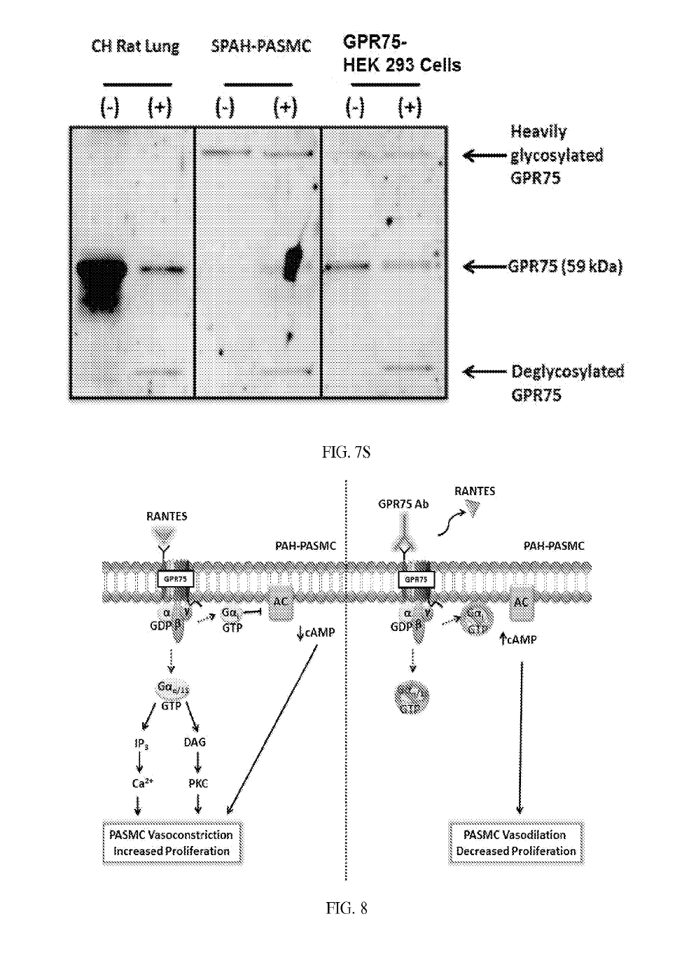

GPR75 is the other GPCR whose mRNA is uniquely expressed in both IPAH- and SPAH-PASMCs. Consistent with this result, in PASMC from each SPAH-patient there was at least an 8-fold increase in protein expression of GPR75 compared to control and in PASMC from each IPAH-patient there was at least a 15-fold increase compared to control (FIG. 7A).

To visualize GPR75 expression, immunofluorescence using a GPR75 N-terminal binding antibody (without permeabilizing the cells) was performed. FIG. 7B shows a prominent increase in GPR75 expression, in IPAH-PASMC compared to Ctrl-PASMC.

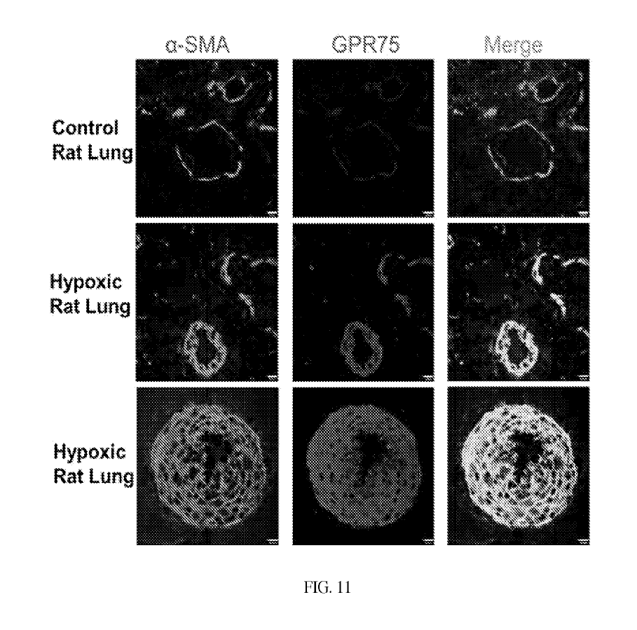

To determine if the increase in GPR75 expression with PAH-PASMC also occurs in animal models of PAH, GPR75 expression was evaluated in lungs from CH mice, CH rats, and MCT-treated rats. FIGS. 7C-I show GPR75 mRNA and protein expression increases in each of the animal models of PAH, compared to controls. GPR75 expression also increased in hearts from the CH rats compared to controls (FIG. 7J), showing increased GPR75 with right ventricular hypertrophy Immunofluorescence of lung tissue further showed that GPR75 expression increased in the smooth muscle layer (detected by staining for α-smooth muscle actin) of the pulmonary arteries of CH rats with pulmonary hypertension compared to control rat lungs (FIG. 11).

### GPR75 Function

GPR75 is an orphan receptor whose endogenous ligand is currently unknown and about which little information is available. Because its mRNA and protein expression is increased in PAH-PASMC and in the lungs of PAH animal models compared to control. An antibody that has been generated against the N-terminal of the receptor (accessible from outside the cell) was tested to determine if it might “block” the receptor. The antibody was obtained and is commercially available from FabGennix Inc. International (Catalog No. GPCR75-101AP). It was reasoned that this antibody, but not an antibody directed at cytoplasm-exposed domains, e.g., intracellular loops or the C-terminal, might block GPR75. Immunoglobulin (IgG) was used as a control. cAMP accumulation and proliferation in PASMCs was then evaluated to determine the role of GPR75 in the function of PAH-PASMCs.

The antibody was added at 1 μg/mL for 2 hrs prior to 10 min stimulation with forskolin (FSK). The N-terminal GPR75 antibody did not significantly alter FSK-induced cAMP accumulation in Ctrl-PASMC compared to the IgG-control (FIG. 7K). However, in IPAH-PASMCs, GPR75 antibody significantly increased FSK-induced cAMP accumulation compared to the IgG-control. These data suggest that the N-terminal GPR75 antibody selectively “blocks” this receptor on IPAH-PASMCs and that GPR75 is coupled to Gαᵢ. The results also showed lower cAMP levels in IPAH-PASMCs compared to Ctrl-PASMCs, which has been shown in previous literature.¹¹

After obtaining the results in FIG. 7K, the effect of the GPR75 antibody on the proliferation of PAH-PASMCs. In order to confirm that the N-terminal GPR75 antibody, the effects were compared to that of a GPR75 antibody targeted to cytoplasmic-exposed domains of the receptor. FIG. 7L shows that proliferation of Ctrl-PASMCs is not significantly changed by addition of control IgG or either the N-terminal-targeted or cytoplasmic domain-targeted GPR75 antibodies. However, in IPAH-PASMCs there is a statistically significant decrease in proliferation upon addition of the N-terminal GPR75 antibody, but not the cytoplasmic-domain binding GPR75 antibody or the IgG-control (FIG. 7L). Because the cytoplasmic-domain binding GPR75 antibody had no effect on the proliferation of PAH-PASMCs, this result provides evidence that the N-terminal binding antibody is having its effect by binding to and blocking activation of the GPR75 receptor on IPAH-PASMCs.

In order to further investigate the function of GPR75, the receptor was overexpressed in heterologous cells so as to mimic its increased expression in IPAH-PASMCs. Primary cells, such as PASMCs, are difficult to transfect due to their membrane integrity; therefore initial experiments were performed in Human Embryonic Kidney (HEK) 293 cells. HEK 293 cells were transfected with a pCMV6-Entry Vector (4.9 kb) alone or vector with a GPR75 insert (1.6 kb); the plasmid is shown in FIG. 9B. The size and sequence of GPR75 was verified by restriction digest (restriction map of GPR75 is shown in FIG. 9C) using restriction enzyme Bgl II and performing agarose gel electrophoresis (FIG. 9D). FIG. 7N shows the expression of GPR75 in HEK 293 cells by western blot after 24, 48, and 72 hrs. The 72 hr time point showed the largest increase in GPR75 compared to cells transfected with empty vector.

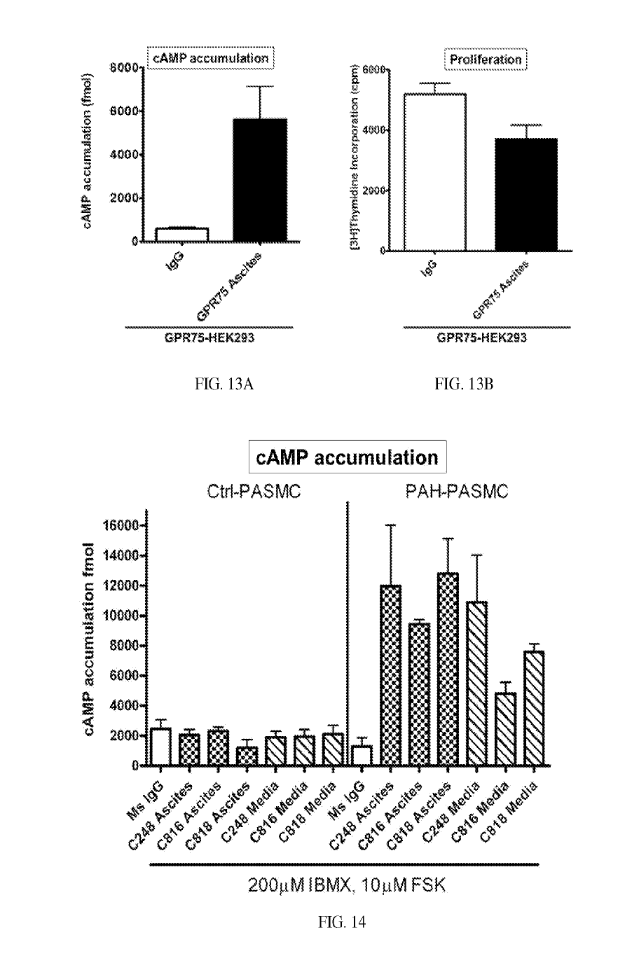

HEK cells engineered to overexpress GPR75 (72 hrs-transfected cells) had significantly lower cAMP levels compared to cells transfected with the empty vector control (FIG. 7O). These data provide further evidence that GPR75 is Gαᵢ-coupled because overexpression lowers basal and forskolin-stimulated levels of cAMP. The results also suggest that GPR75 may be constitutively active, i.e., has activity in the absence of agonist, since basal cAMP levels are lower in the cells transfected with the GPR75 vector than with control vector. It has been found that receptors may exist in a constitutively active state, particularly when expressed in high amounts in cultured cells. Alternatively, an agonist for the receptor may be present in the culture media or released by the cells in an autocrine manner. N-terminal-targeted GPR75 polyclonal antibody (FIG. 7M) and ascites fluid obtained from mice that produced an N-terminal directed GPR75 monoclonal antibody (but not control IgG) increased cAMP accumulation in HEK293 cells engineered to express GPR75 (FIG. 13A). The latter results imply that these antibodies inhibit the constitutive activity of GPR75.

GPR75 overexpressed HEK 293 cells also show a significant increase in proliferation compared to empty vector-transfected cells (FIG. 7Q).

GPR75-expressing HEK 293 cells also have increased DNA synthesis compared to empty vector-transfected cells (FIG. 7Q). Ascites fluid obtained from mice that produced an N-terminal directed GPR75 mAb not only increases cAMP accumulation in the GPR75-expressing cells (FIG. 13B) but also decreases their DNA synthesis (FIG. 13B), providing further evidence for the ability of GPR75 to promote cell proliferation and that GPR75 is active but can be inhibited by an antibody directed to the N-terminal region of the receptor.

PASMCs were transfected using electroporation with an Amaxa nucleofector device to express GPR75 (FIG. 9E).

### GPR75 is Glycosylated

Detection of GPR75 by Western Blot often revealed, double or triple bands (FIG. 7R). Such patterns often occur as a consequence of receptor glycosylation.

A deglycosylation assay was performed to demonstrate that GPR75 is glycosylated and to investigate if its migration pattern changed. FIG. 7S shows a Western Blot of CH rat lung, SPAH-PASMCs, and GPR75 vector-transfected HEK 293 cell lysate before and after the addition of Peptide-N-Glycosidase F (PNGase F, an amidase that cleaves between the innermost GlcNAc and asparagine residues of high mannose, hybrid, and complex oligosaccharides from N-linked glycoproteins). FIG. 7S shows a lower band that appears in the presence of PNGase F The upper most band is likely a more heavily glycosylated receptor that is not fully deglycosylated, the middle band being the glycosylated receptor (at the appropriate 59 kDa size) while the lowest band likely represents a deglycosylated form of the receptor.

These data suggest that GPR75 is glycosylated. Interestingly the GPR75 receptor seems to change in glycosylation state in PAH, as shown for IPAH-PASMC and CH rat hearts (FIGS. 7A and 7J).

### Results Part VI

Circulating Microparticles in Humans and its Expression is Increased in Microparticles from Patients with PAH

In embodiments, microparticles can be isolated from the peripheral blood by centrifugation of plasma. PAH (PAH) subjects have higher circulating microparticle protein levels (FIG. 10A). GPR75 protein is detected in these microparticles and is at higher levels in microparticles isolated from the blood of patients with PAH (FIG. 10B).

### Discussion of Results

PAH is characterized by increased pulmonary vascular resistance, in part due to enhanced vasoconstriction and increased proliferation of PASMCs. Finding unique GPCRs in PAH-PASMCs through expression profiling that compares normal and diseased patient-derived cells could be useful for uncovering new targets for PAH in the disease. Unbiased approaches have begun to identify GPCR expression in specific tissues. The quantification of RNA transcripts for 353 non-odorant GPCRs in 41 tissues from mice revealed new roles for a number of GPCRs in various tissues.⁴⁰ Many orphan GPCRs (receptors whose endogenous agonist ligand is not known) and olfactory GPCRs are expressed in tissues, but their function has yet to be determined.⁴¹ Limited data are available regarding GPCR expression in individual cells, which can express >100 GPCRs.⁴⁴ Because the GPCR profile of PASMC has not been identified, key GPCRs that regulate pulmonary circulation may have been overlooked.

Microarray data, such as those by Affymetrix, that assess total cellular mRNA are not optimal for detecting the expression of GPCRs. A specific TAQMAN GPCR array was therefore used to investigate GPCR expression in control- SPAH- and IPAH-PASMC. Microarrays are one of the leading methods to identify differentially expressed genes, however their reliability in detecting differences in RNA expression hinges on many factors. These factors include RNA extraction, probe labeling, hybridization conditions, as well as array production. Due to such limitations in reliability, mRNAs identified as differentially expressed on the gene array need to be validated with other methods.⁴⁵ Results obtained from the GPCR array were validated by independent real-time PCR to confirm the relative expression of GPCRs identified from the array and to focus future studies. Real-time PCR is quantitative, requires a low amount of RNA, is relatively inexpensive, and provides rapid results.^{45,46} Data showed that the GPCR array, with data confirmed by independent real-time PCR (using primers designed in the lab), provides a reliable tool (based on Cₜ values) to determine GPCR expression in cells (Table 9).

Since mRNA expression may not correlate with protein expression or function, the approaches described herein sought to identify GPCRs that are functional in PASMC. It was found, as a result of use of these approaches, that receptor expression correlates with cAMP production and function (e.g., increases in cAMP accumulation and DNA synthesis, a measure of cell proliferation). The findings of the vasoactive intestinal peptide receptor 1 (VIPR1, Gαₛ-coupled) and the oxytocin receptor (Gαi-coupled) being highly expressed in PASMCs establish their role in PASMCs. The data show that in PASMC_VIPR1 protein is expressed in the membrane fraction of PASMCs and that its activation by VIP increases cAMP accumulation and decreases their proliferation by roughly 50% (0.1 nM-10 μM VIP, FIG. 4C). In parallel, oxytocin receptor protein was found to be expressed on membranes of PASMC and that oxytocin dose-dependently (0.1 nM-10 μM, 10 min) decreased cAMP accumulation and promoted PASMC proliferation (in cells grown in serum free media over 24 hr (FIG. 4D)).

Expression and function of VIPR1 in PASMC is consistent with recent findings indicating that VIP could be important in pulmonary artery remodeling and even beneficial in the treatment of PAH. VIP (−/−) mice spontaneously develop moderate pulmonary remodeling, variants in the VIP gene occur in IPAH and chronic inhalation of VIP was shown to improve the hemodynamics and exercise capacity in a small (n=8) cohort of PAH-patients.⁴⁷⁻⁴⁹ The expression and function of oxytocin receptors in PASMCs corresponds to previous studies that found oxytocin (0.2 units/kg) increases mean PAP and pulmonary vascular resistance (PVR). Interestingly cytokines, such as IL-6, which are up-regulated in PAH increase the expression of the oxytocin receptor.⁵⁰ Thus, the data summarized herein validates the use of a GPCR-array as an initial approach to discover highly expressed or unrecognized GPCRs in PASMCs that may contribute to the physiology and pathophysiology of the pulmonary vasculature.

The GPCR microanay, in addition to profiling GPCR expression in a specific cell type, identified uniquely expressed receptors in PAH-PASMCs as potential therapeutic targets for PAH. Orphan receptors can be difficult to study as no agonists or antagonists are available for functional studies. Methods to investigate their signaling and functional role include RNA knockdown, generation of receptor-knockout mice and is of antibodies that can bind receptors and block function (or promote their loss from the cell surface).

Two orphan GPCRs were identified that are uniquely expressed in both IPAH-PASMCs and SPAH-PASMCs compared to control, namely GPR113 and GPR75. Both might be therapeutic targets for PAH. As orphan receptors, GPR113 and GPR75 lack an identified endogenous agonist. Limited research has been done on GPR113: only 5 articles have been published regarding this receptor. The gene for GPR113 maps to chromosome 2p23.3 and makes up a rather large 1,079 amino acid protein (116,341 Da). GPR113 is an adhesion GPCR belonging to family 2B, a family characterized by receptors having long N-terminal extracellular domains. Its expression was thought to be restricted to a subset of taste receptor cells.^{51,52} It has a GPCR proteolytic site (GPS) domain in its N-terminus and long Ser/Thr-rich regions forming mucin-like stalks. GPR113 has a hormone binding domain and one epidermal growth factor (EGF) domain.⁵³ No commercially available antibodies that were tested allowed assessment of the function of GPR113. In past studies, GPR113 was found to be up-regulated >5-fold in small bowel neuroendocrine tumors compared to normal tissues and thus, perhaps it may be associated with other disease states.⁵⁴

This application describes the discovery that PAH is associated with increased expression of GPR75. To date only 4 articles have been published regarding GPR75. The gene for GPR75 maps to chromosome 2p16 and encodes a 540 amino acid protein (59,359 Da).⁵⁵ GPR75 is highly expressed in the retina and central nervous system.⁵⁶ It has been proposed that Regulated upon Activation, Normal T-cell Expressed, and Secreted (RANTES, Chemokine Ligand 5 [CCL5]) may be a ligand for GPR75. Upon treatment with RANTES, an increase in inositol trisphosphate (IP3), and stimulation of Ca²⁺ mobilization was noted and treatment with U73122 (a PLC inhibitor) blocked Ca²⁺ mobilization, suggesting that GPR75 couples to Gα_{q/11}.⁵⁷ RANTES is a chemokine that recruits leukocytes into inflammatory sites and also induces the proliferation and activation of natural-killer cells. RANTES levels are increased in patients with severe PAH⁵⁹. Sequence-structure based phylogeny predicted that Neruopeptide Y may be a potential ligand for GPR75⁶⁰. Neuropeptide Y stimulates proliferation of human PASMCs⁶¹.

PAH-PASMCs are known to produce lower basal levels of cAMP than do Ctrl-PASMCs, but the effect of GPR75 and assess its effect on cAMP accumulation in both Ctrl- and IPAH-PASMCs has not been evaluated.¹¹ A commercially available N-terminal antibody was used as a type of antagonist to “block” receptor activation. Antibodies that target intracellular domains with intact cells cannot be used for this purpose as they would need to get into the cell to be functional. An N-terminal GPR75 antibody was found to increase cAMP accumulation and decrease proliferation of PAH-PASMCs, but not control-PASMCs that have lower expression of GPR75. The results suggest that GPR75 is a Gαᵢ-coupled GPCR, in addition to being previously shown as a Gα_{q/11}-coupled.⁵⁸ A program, PRED-COUPLE2, is a tool that uses amino acid sequence of a GPCR to predict its coupling to G proteins by using a refined library of highly-discriminative Hidden Markov Models. Hits from individual profiles are combined by a feed-forward artificial neural network to produce the final output. Query of the GPR75 sequence predicted its G protein-coupling specificity as follows (on a 1.00 scale): Gαi=0.99, Gαq=0.28, Gαl2/13=0.11 and Gαs=0.02, which is consistent with the experimental results that indicate coupling of GPR75 to Gαi.

HEK293 cells engineered to express GPR75 recapitulate the signaling and functional responses of PAH-PASMCs: decrease in cAMP accumulation and inhibition of DNA synthesis. Such HEK293 cells are potentially useful to screen and identify GPR75-interacting drugs and antibodies as an assay system with functional readouts for discovery and validation of therapeutics for PAH that target GPR75.

Treatment with the N-terminal directed antibody receptor increases cAMP levels (FIGS. 7P, 12, 13A, 14), which is characteristic of inhibition of a Gαi-coupled GPCR, and decreases DNA synthesis of PAH-PASMCs (FIG. 7M) and of HEK293 cells expressing GPR75 (FIG. 13B). Expressing GPR75 in HEK293 cells lowers basal cAMP levels and increases DNA synthesis of the cells (FIGS. 7O-P). An antibody to GPR75 blunts both these responses (FIG. 13), results providing further evidence that GPR75 couples to Gαi.

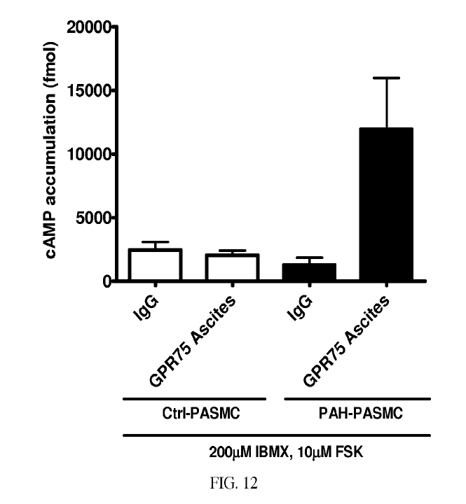

The initial studies here used an N-terminal directed polyclonal antibody to study PAH-PASMCs and GPR75-expressing HEK 293 and indicated that this “blocking” antibody can restore the phenotype of PAH-PASMCs to that of PASMCs from control subjects, in terms of cAMP accumulation and DNA synthesis. To develop a more therapeutically relevant approach, monoclonal antibodies (mAbs) were generated. The use of mAbs is more advantageous than polyclonal sera because mAbs bind to their targets with high specificity, have less cross-reactivity with other proteins, and therefore have excellent potential and established utility as therapeutic agents. Custom monoclonal antibodies were generated with specificity to a particular epitope (amino acid sequence: PNATSLHVPHSQEGNSTS (SEQ ID NO:2)-NH2) in the N-terminal (extracellular) domain of the GPR75 protein. Antibodies were generated in mice. Tests of the ascites fluid from seven mice yielded 3 that recognized GPR75 on western blots. Those 3 hybridomas were cultured and their conditioned media tested for ability to raise cAMP in PAH-PASMCs and in GPR75-expressing HEK-293 cells and to effect DNA synthesis in the latter cells. The N-terminal directed GPR75 mAbs increased cAMP accumulation in PAH-PASMCs (FIGS. 12, 14) and in GPR75-expressing HEK 293 (FIG. 13A). Furthermore, the mAbs decreased proliferation in the latter cells (FIG. 13B). Thus, GPR75-targetted mAbs block GPR75 signaling and function and have the potential to be therapeutic agents for the treatment of PAH. The ability to detect increased GPR75 in microparticles from the blood of patients with PAH (FIG. 10B) provides a potential way to stratify patients with PAH in terms of their GPR75 expression and thus to identify and monitor patients with this disease.

FIG. 8 is a schematic of GPR75 signaling, either via RANTES⁵⁹ or when blocked by an N-terminal binding GPR75 antibody. Previous literature has shown that antibodies can block the activity of membrane proteins. For example, blocking IL-17A with anti-IL-17A antibody can protect against lung injury-induced pulmonary fibrosis.⁶² Although antibodies are more expensive to develop than small molecules, they tend to have a longer duration of action. It has been suggested that antibody therapeutics might be possible to develop against ˜88 GPCRs, some of which would require agonistic antibodies.⁶³ Such therapeutic antibodies have shown the greatest success in inflammatory diseases, although some success has been seen in cardiovascular diseases.⁶⁴ It has been shown that sometimes targeting the receptor can be more successful than targeting the ligand with an antibody. This has been shown by the observed lack of efficacy when targeting MIP1-α or RANTES (CCL5) as opposed to targeting the receptors CCR1 and CCR5.⁶¹ The same is true for CXCL8 (IL-8) and its receptors CXCR1 and CXCR2.⁶⁵ Ligand levels can increase to overcome antibody blockade of such ligands easier than expression of receptors can increase. Also there is a redundancy of some GPCRs for multiple ligands.⁶⁶ Antibodies directed towards GPCRs can play a therapeutic role not only by altering signaling pathways, such as those involved in proliferation and vasodilation in PAH, but also by serving as carriers for targeted toxin therapy.⁶⁰ Therefore, embodiments relate to using an anti-GPR75 antibody in therapeutic treatment of patients with IPAH and SPAH based on the much higher GPR75 expression in PAH-PASMCs relative to Ctrl-PASMCs. Blocking GPR75 is predicted to block whichever G protein or other pathways this receptor uses to perturb cell function and in the setting of PAH, to promote vasodilation and decreased PASMC proliferation. Further research into the expression of GPR75 in other cell types within the pulmonary and systemic circulation could help predict adverse effects. Drug and/or small molecule screening could also be beneficial to determine potential ligands for the orphan GPCRs that are now believe to have an important role in the pathogenesis of PAH: GPR113 and GPR75 or perhaps other orphans highlighted in Tables 14 and 15, each of which is contemplated as being used in the present methods of diagnosis and treatment.

GPR75 has multiple glycosylation sites. Glycosylation can influence the activity of a receptor and hence could contribute to PAH. GPR75 has 3 putative N-glycosylation sites (at position 2, 12, and 25). Results here with PNGase F strongly suggest that GPR75 is glycosylated (FIG. 7S). Receptor glycosylation is often necessary to transport a receptor to the cell surface and can stabilize receptors there.⁶⁷ Glycosylated receptors, in particular complex species generated from the high mannose form, are considered more “mature”.⁶⁸ Glycosylation can influence receptor-ligand binding.^{69,70} Glycosylation can also be essential for conformational changes required for G protein coupling and subsequent signaling. Some GPCRs show blunted cAMP levels if the receptors in not properly glycosylated.⁶⁷ Glycosylation of GPR75 thus may contribute to its signaling. Deglycosylation strategies might be able to influence receptor activity in PAH-PASMCs.

Cellular microparticles (MPs), a heterogeneous population of vesicles with a diameter of 100-1000 nm, are derived from membrane shedding. MPs vary in size, composition and function. Increased circulating MPs occur in PAH patients compared to controls (FIG. 10)⁷¹⁻⁷³. GPR75 expression can be detected on circulating MPs and its expression is greater in PAH patient-derived than in control MPs (FIG. 10B). These results imply that circulating GPR75-MPs could serve as a biomarker to identify and monitor patients with PAH and thus have important clinical utility in terms of improved ability to detect PAH, which can be “silent” in early stages of the disease. Early detection can facilitate early intervention and modification of its progression.

In summary, GPCR arrays are useful for profiling cellular GPCR expression and discovering GPCRs that regulate PASMCs and can contribute to the pathophysiology of PAH. Profiling GPCRs in PAH patients may aid in the development of personalized medicine strategies by revealing GPCRs uniquely expressed in IPAH compared to SPAH (Tables 14 and 15). A second personalized medicine approach involves assessment of circulating microparticles (FIG. 10). GPCR analysis can identify receptors for the treatment of PAH in specific patients. The discovery of novel GPCRs in PAH identifies targets and ultimately drugs that might rapidly enter clinical trials since many GPCR agonists/antagonists are approved for clinical use. Orphan GPCRs provide new targets for which antibodies, such as mAbs to GPR75 or GPR113, may be administered in a therapeutic approach described in embodiments herein to produce a paradigm shift in the treatment of PAH.

Having described various embodiments of the invention above, it will be apparent to those skilled in the art that modifications and variations are possible without departing from the scope of the disclosure as defined in the appended claims.

Various publications, including patents, published applications, technical articles and scholarly articles are cited throughout the specification. Each of these cited publications is incorporated by reference herein, in its entirety.

### CITED REFERENCES

1. Fishman A P. (1998) Etiology and pathogenesis of primary pulmonary hypertension: a perspective. Chest; 114: 242S-247S.
2. Rabinovitch M. Molecular pathogenesis of pulmonary arterial hypertension. J Clin Invest 2008; 118:2372-9.
3. Humbert M, Morrell N W, Archer S L et al (2004) Cellular and molecular pathobiology of pulmonary arterial hypertension. J Am Coll Cardiol 43:13S-24S.
4. Gaine S P, and Rubin L J. (1998) Primary pulmonary hypertension. Lancet. 352: 719-725.
5. Rabinovitch M. Molecular pathogenesis of pulmonary arterial hypertension. J Clin Invest. 2012 Dec. 3; 122(12):4306-13.
6. Klings E S, Farber H W. (2001). Current management of primary pulmonary hypertension. Drugs. 61(13):1945-56.
7. Wanstall J C, Jeffery T K. (1998). Recognition and management of pulmonary hypertension. Drugs. 56(6):989-1007.
8. Archer S, Rich S. (2000) Primary pulmonary hypertension: a vascular biology and translational research “Work in progress”. Circulation. 102(22):2781-91.
9. Sutherland E W, Rall T W. Fractionation and characterization of a cyclic adenine ribonucleotide formed by tissue particles. J Biol Chem. 1958 June; 232(2):1077-91.
10. Insel P A, Zhang L, Murray F, Yokouchi H, Zambon A C. Cyclic AMP is both a pro-apoptotic and anti-apoptotic second messenger. Acta Physiol (Oxf). 2012 February; 204(2):277-87.
11. Murray F, Maclean M R, Insel P A. Role of phosphodiesterases in adult-onset pulmonary arterial hypertension. Handb Exp Pharmacol. 2011; (204):279-305.
12. Lerner, A., Epstein, P. M. (2006). Cyclic nucleotide phosphodiesterases as targets for treatment of haematological malignancies. Biochem. J. 393, 21-41.
13. Creighton J R, Masada N, Cooper D M, Stevens T. Coordinate regulation of membrane cAMP by Ca2+-inhibited adenylyl cyclase and phosphodiesterase activities. Am J Physiol Lung Cell Mol Physiol 2003; 284: L100-7.
14. Jourdan K B, Mason N A, Long L, Philips P G, Wilkins M R, Morrell N W. Characterization of adenylyl cyclase isoforms in rat peripheral pulmonary arteries. Am J Physiol Lung Cell Mol Physiol 2001; 280:L1359-69.
15. Beavo J A, and Brunton L L. (2002) Cyclic nucleotide research—still expanding after half a century. Nat Rev Mol Cell Biol. 3(9):710-718.
16. Conti M, Beavo J. (2007). “Biochemistry and physiology of cyclic nucleotide phosphodiesterases: essential components in cyclic nucleotide signaling.” Annu Rev Biochem. 76:481-511.
17. Pierre S, Eschenhagen T, Geisslinger G, and Scholich K. (2009) “Capturing adenylyl cyclases as potential drug targets.” Nature Reviews Drug Discovery. 8: 321-335.
18. Murray K J. (1990). Cyclic AMP and mechanism of vasodilation. Pharmacol Ther. 47: 329-345.
19. Della Fazia M A, Servillo G, Sassone-Corsi P. (1997). Cyclic AMP signalling and cellular proliferation: regulation of CREB and CREM. FEBS Lett. 410:22-24.
20. Koyama H, Bornfeldt K E, Fukumoto S and Nishizawa Y. (2001). Molecular pathways of cyclic nucleotide-induced inhibition of arterial smooth muscle cell proliferation. J. Cell Physiol. 186:1-10.
21. Southgate K and Newby A C. (1990). Serum-induced proliferation of rabbit aortic smooth muscle cells from the contractile state is inhibited by 8-Br-cAMP but not 8-Br-cGMP. Atherosclerosis. 82: 113-123.
22. Cornwell T L, Arnold E, Boerth N J and Lincoln T M. (1994) Inhibition of smooth muscle cell growth by nitric oxide and activation of cAMP-dependent protein kinase by cGMP. Am. J. Physiol. 267: C1405-C1413.
23. Grosser T, Bonisch D, Zucker T P and Schror K. (1995). Iloprost-induced inhibition of proliferation of coronary artery smooth muscle cells is abolished by homologous desensitisation. Agent Action Suppl. 45: 58-91.
24. Kronemann N, Nocher W A, Busse R, Schini-Kerth V B. (1999). Growth-inhibitory effect of cyclic GMP- and cAMP-dependent vasodilators on rat vascular smooth muscle cells: effect on cell cycle and cyclin expression. Br J Pharmacol. 126: 349-357.
25. Hamad A M, Range S P, Holland E, and Knox A J. (1999). Desensitization of guanylyl cyclases in cultured human airway smooth-muscle cells. Am. J. Respir. 20: 1087-1095.
26. Graves L M, Bomfeldt K E, Raines E W, Potts B C, Macdonald S G, Ross R, Krebs E G. (1993). Protein kinase A antagonises platelet-derived growth factor-induced signalling by mitogen-activated protein kinase in human arterial smooth muscle cells. Proc Natl Acad Sci. 90: 10300-10304.
27. Bomfeldt K E, and Krebs E G. (1999). Crosstalk between protein kinase A and growth factor receptor signalling pathways in arterial smooth muscle. Cell Signal. 11: 465-477.
28. Bonisch D, Weber A A, Wittpoth M, Osinski M T, and Schror K. (1998). Antimitogenic effects of trapidil in coronary artery smooth muscle cells by direct activation of protein kinase A. Mol Pharmacol. 54: 241-248.
29. Zucker T P, Bonisch D, Hasse A, Grosser T, Weber A A, and Schror K. (1998). Tolerance development to antimitogenic actions of prostacyclin but not prostaglandin E1 in coronary artery smooth muscle cells. Eur J. Pharmacol. 345: 213-220.
30. Graves L M, Bornfeldt K E, Argast G M, Krebs E G, Kong X, Tin T A, Lawrence J C Jr. (1995). cAMP- and rapamycin-sensitive regulation of the association of eukaryotic initiation factor 4E and the translational regulator PHAS-I in aortic smooth muscle cells. Proc Natl Acad Sci. 92: 7222-7226.
31. Schmidt M, Dekker F J, Maarsingh H. Exchange Protein Directly Activated by cAMP (epac): A Multidomain cAMP Mediator in the Regulation of Diverse Biological Functions. Pharmacol Rev. 2013 Feb. 27; 65(2):670-709.
32. Wu J, Dent P, Jelinek T, Wolfman A, Weber M J, Sturgill T W. (1993) Inhibition of the EGF activated MAP kinase signalling pathway by adenosine 3′,5′-monophosphate. Science 262: 1065-1069.
33. Hafner S, Adler H S, Mischak H, Janosch P, Heidecker G, Wolfman A, Pippig S, Lohse M, Ueffing M, Kolch W. (1994). Mechanism of inhibition of Raf-1 by protein kinase A. Mol Cell Biol. 14: 6696-6703.
34. Bos J L. Epac proteins: multi-purpose cAMP targets. Trends Biochem Sci. 2006 December; 31(12): 680-6.
35. Yokoyama U, Minamisawa S, Quan H, Akaike T, Suzuki S, Jin M, Jiao Q, Watanabe M, Otsu K, and Iwasaki S, et al. (2008) Prostaglandin E2-activated Epac promotes neointimal formation of the rat ductus arteriosus by a process distinct from that of cAMP-dependent protein kinase A. J Biol Chem 283:28702-28709.
36. Tang C M, Insel P A. GPCR expression in the heart; “new” receptors in myocytes and fibroblasts. Trends Cardiovasc Med 2004; 14:94-9.
37. Wu J, Dent P, Jelinek T, Wolfman A, Weber M J, Sturgill T W. (1993) Inhibition of the EGF-activated MAP kinase signalling pathway by adenosine 3′,5′-monophosphate. Science 262: 1065-1069.
38. Dupre D J, Robitaille M, Rebois R V, Hebert T E. The Role of Gbetagamma Subunits in the Organization, Assembly, and Function of GPCR Signaling Complexes. Annu Rev Pharmacol Toxicol 2009; 49:31-56.
39. Marinissen M J, Gutkind J S. G-protein-coupled receptors and signaling networks: emerging paradigms. Trends Pharmacol Sci 2001; 22:368-76.
40. Regard J B, Sato I T, Coughlin S R. Anatomical profiling of G protein-coupled receptor expression. (2008) Cell 135(3):561-571.
41. Hakak Y, Shrestha D, Goegel M C, Behan D P, Chalmers D T. Global analysis of G-protein-coupled receptor signaling in human tissues. (2003) FEBS Lett. 550(1-3):11-17.
42. Shimoda L A, Laurie S S. Vascular remodeling in pulmonary hypertension. J Mol Med (Berl). 2013 March; 91(3):297-309.
43. Morimatsu Y, Sakashita N, Komohara Y, Ohnishi K, Masuda H, Dahan D, Takeya M, Guibert C, Marthan R. Development and characterization of an animal model of severe pulmonary arterial hypertension. J Vasc Res. 2012; 49(1):33-42.
44. Insel P A, Snead A, Murray F, Zhang L, Yokouchi H, Katakia T, Kwon O, Dimucci D, Wilderman A. GPCR expression in tissues and cells: are the optimal receptors being used as drug targets? Br J Pharmacol. 2012 March; 165(6):1613-6.
45. Rajeevan M. S., Ranamukhaarachchi, D. G., Vernon, S. D., Unger, E. R. Use of Real-Time Quantitative PCR to Validate the Results of cDNA Array and Differential Display PCR Technologies. (2001). Methods 25, 443-451.
46. Boyd, L. M., Richardson, W. J., Chen, J., Kraus, V. B., Tewari, A., Setton, L. A. Osmolarity Regulates Gene Expression in Intevertebral Disc Cells Determined by Gene Array and Real-Time Quantitative RT-PCR. (2005). Annals of Biomedical Engineering 33, 1071-1077.
47. Haberl I, Frei K, Ramsebner R, Doberer D, Petkov V, Albinni S, Lang I, Lucas T, Mosgoeller W. Vasoactive intestinal peptide gene alterations in patients with idiopathic pulmonary arterial hypertension. Eur J Hum Genet. 2007; 15(1):18-22.
48. Said S I, Hamidi S A, Dickman K G, Szema A M, Lyubsky S, Lin R Z, Jiang Y P, Chen J J, Waschek J A, Kort S. Moderate pulmonary arterial hypertension in male mice lacking the vasoactive intestinal peptide gene. Circulation. 2007; 115(10):1260-1268.
49. Zhang Y, Zhang J Q, Liu Z H, Xiong C M, Ni X H, Hui R T, He J G, Pu J L. VIP gene variants related to idiopathic pulmonary arterial hypertension in Chinese population. Clin Genet. 2009; 75(6):544-549.
50. Roberts N V, Keast P J, Brodeky V, Oates A, Ritchie B C. The effects of oxytocin on the pulmonary and systemic circulation in pregnant ewes. Anaesth Intensive Care. 1992; 20(2):199-202.
51. LopezJimenez N D, Sainz E, Cavenagh M M, Cruz-Ithier M A, Blackwood C A, Battey J F, Sullivan S L. Two novel genes, Gpr113, which encodes a family 2 G-protein-coupled receptor, and Trcg1, are selectively expressed in taste receptor cells. Genomics. 2005 April; 85(4):472-82.
52. Bjarnadóttir T K, Fredriksson R, Höglund P J, Gloriam D E, Lagerström M C, Schiöth H B. The human and mouse repertoire of the adhesion family of G-protein-coupled receptors. Genomics. 2004 July; 84(1):23-33.
53. Fredriksson R, Lagerström M C, Hoglund P J, Schiöth H B. Novel human G protein-coupled receptors with long N-terminals containing GPS domains and Ser/Thr-rich regions. FEBS Lett. 2002 Nov. 20; 531(3):407-14.
54. Carr J C, Boese E A, Spanheimer P M, Dandaleh F S, Martin M, Calva D, Schafer B, Thole D M, Braun T, O'Dorisio T M, O'Dorisio M S, Howe J R. Differentiation of small bowel and pancreatic neuroendocrine tumors by gene-expression profiling. Surgery. 2012 December; 152(6):998-1007.
55. Tarttelin E E, Kirschner L S, Bellingham J, Baffi J, Taymans S E, Gregory-Evans K, Csaky K, Stratakis C A, Gregory-Evans C Y. Cloning and characterization of a novel orphan G-protein-coupled receptor localized to human chromosome 2p16. Biochem Biophys Res Commun. 1999 Jun. 24; 260(1):174-80.
56. Sauer C G, White K, Stohr H, Grimm T, Hutchinson A, Bernstein P S et al. (2001). Evaluation of the G protein coupled receptor-75 (GPR75) in age related macular degeneration. Br J Ophthalmol 85: 969-975.
57. Ignatov A, Robert J, Gregory-Evans C, Schaller H C. RANTES stimulates Ca2+ mobilization and inositol trisphosphate (IP3) formation in cells transfected with G protein-coupled receptor 75. Br J Pharmacol. 2006 November; 149(5):490-7.
58. Liu B, Hassan Z, Amisten S, King A J, Bowe J E, Huang G C, Jones P M, Persaud S J. The novel chemokine receptor, G-protein-coupled receptor 75, is expressed by islets and is coupled to stimulation of insulin secretion and improved glucose homeostasis. Diabetologia. 2013 November; 56(11):2467-76.
59. Dorfmüller P, Zarka V, Durand-Gasselin I, Monti G, Balabanian K, Garcia G, Capron F, Coulomb-Lherminé A, Marfaing-Koka A, Simonneau G, Emilie D, Humbert M. Chemokine RANTES in severe pulmonary arterial hypertension. Am J Respir Crit Care Med. 2002 Feb. 15; 165(4):534-9.
60. Kakarala and Jamil Sequence-structure based phylogeny of GPCR Class A Rhodopsin receptors. Mol Phylogenet Evol. 2014 May; 74:66-96.
61. Crnkovic S, Egemnazarov B, Jain P, Seay U, Gattinger N, Marsh L M, Bálint Z, Kovacs G, Ghanim B, Klepetko W, Schermuly R T, Weissmann N, Olschewski A, Kwapiszewska G. NPY/NPY1R mediated vasoconstrictory and proliferative effects in pulmonary hypertension. Br J Pharmacol. 2014 Apr. 30.
62. Mi S, Li Z, Liu H, Hu Z W, Hua F. [Blocking IL-17A protects against lung injury-induced pulmonary fibrosis through promoting the activation of p50NF-kappaB]. Yao Xue Xue Bao. 2012 June; 47(6):739-44.
63. Hutchings, C. J., Koglin, M., Marshall, F. H. Therapeutic antibodies directed at G protein coupled receptors. (2010). MAbs2, 594-606.
64. Grader M, Shankar G, Goetzl E J. Cutting edge: suppression of T cell chemotaxis by sphingosine 1-phosphate. J Immunol 2002; 169:4084-7.
65. Goetzl E J, Dembrow D, Van Brocklyn J R, Graler M, Huang M C. An IgM-kappa rat monoclonal antibody specific for the type 1 sphingosine 1-phosphate G protein-coupled receptor with antagonist and agonist activities. Immunol Lett 2004; 93:63-9.
66. Hancock W W. Chemokines and transplant immunobiology. J Am Soc Nephrol 2002; 13:821-4.
67. Yan Y, Scott D J, Wilkinson T N, Ji J, Tregear G W, Bathgate R A. Identification of the N-linked glycosylation sites of the human relaxin receptor and effect of glycosylation on receptor function. Biochemistry. 2008 Jul. 1; 47(26):6953-68.
68. Tansky M F, Pothoulakis C, Leeman S E. Functional consequences of alteration of N-linked glycosylation sites on the neurokinin 1 receptor. Proc Natl Acad Sci USA. 2007 Jun. 19; 104(25):10691-6.
69. Arey B J, López F J. Are circulating gonadotropin isoforms naturally occurring biased agonists? Basic and therapeutic implications. Rev Endocr Metab Disord. 2011 December; 12(4):275-88.
70. Xu L, Go E P, Finney J, Moon H, Lantz M, Rebecchi K, Desaire H, Mure M. Post-translational Modifications of Recombinant Human Lysyl Oxidase-like 2 (rhLOXL2) Secreted from Drosophila S2 Cells. J Biol Chem. 2013 Feb. 22; 288(8):5357-63.
71. Bakouboula B, Morel O, Faure A, Zobairi F, Jesel L, Trinh A Zupan M, Canuet M, Grunebaum L, Brunette A, Desprez D, Chabot F, Weitzenblum E, Freyssinet J M, Chaouat A, Toti F.Procoagulant, Membrane Microparticles Correlate with the Severityof Pulmonary Arterial Hypertension. Am J Respir Crit Care Med. 2008; 177:536-43.
72. Amabile N, Heiss C, Real W M, Minasi P, McGlothlin D, Rame E J, Grossman W, De Marco T, Yeghiazarians Y. Circulating endothelial microparticle levels predict hemodynamic severity of pulmonary hypertension. Am J Respir Crit Care Med 2008; 177:1268-75.
73. Diehl P, Aleker M, Helbing T, Sossong V, Germann M, Sorichter S, Bode C, Moser M., Increased platelet, leukocyte and endothelial microparticles predict enhanced coagulation and vascular inflammation in pulmonary hypertension. J Thromb Thrombolysis 2011; 31:173-9.

What is claimed is:

1. A method for diagnosing or treating pulmonary arterial hypertension (PAH) in a subject, comprising obtaining a biological sample from said subject, and measuring an expression level of GPR75 in the sample of the subject, wherein an increased expression level of GPR75 in the sample of the subject as compared to a reference level of GPR75 in a control sample provides a diagnosis or an indication for treatment of PAH in the subject.

2. The method of claim 1, wherein said biological sample is selected from the group consisting of blood, plasma, serum, lung cells or tissues, and heart cells or tissues.

3. The method of claim 2, wherein said cells are pulmonary arterial smooth muscle cells (PASMCs).

4. A method for diagnosing or treating pulmonary arterial hypertension (PAH) in a subject in need thereof, comprising obtaining a biological sample from said subject, and measuring an amount of circulating microparticles (MPs) in the sample of the subject, wherein an increased amount of MPs as compared to a reference amount of MPs in a control sample provides a diagnosis or an indication for treatment of PAH in the subject.

5. The method of claim 4, wherein the measuring of the amount of circulating MPs in the sample of the subject comprises detecting GPR75 that is expressed on said MPs.

6. The method of claim 4, wherein said biological sample is selected from the group consisting of blood, plasma, serum, lung cells or tissues, and heart cells or tissues.

7. The method of claim 6, wherein said cells of the biological sample are pulmonary arterial smooth muscle cells (PASMCs).

8. A kit for diagnosing pulmonary arterial hypertension (PAH) in a subject in need thereof, comprising:
a) a capture reagent comprising one or more detectors specific for binding to circulating microparticles (MPs) and/or to GPR75 expressed thereon,
b) a detection reagent specifically reactive with the capture reagent, and
c) instructions for using the kit for diagnosing and providing an indication of a treatment of PAH in said subject when an increased amount of the circulating MPs or an expression level of the GPR75 is detected in a bodily sample of the subject bodily sample as compared to a reference amount of circulating MPs or a reference expression level of GPR75 in a control sample.

9. The kit of claim 8, wherein said bodily sample is selected from the group consisting of blood, plasma, serum, lung cells or tissues, and heart cells or tissues.

10. The kit of claim 9, wherein said cells of the bodily sample are pulmonary arterial smooth muscle cells (PASMCs).

11. A method for treating PAH, comprising:
administering to a subject in need thereof a pharmaceutical composition comprising a therapeutically effective amount of an agent that inhibits GPR75.

12. The method of claim 11, wherein the agent is a GPR75 agonist or antagonist that interacts with the GPR75 expressed by pulmonary arterial smooth muscle cells (PASMCs) of the subject so as to inhibit signaling or function, or an mRNA, DNA, or protein expression level, of the GPR75.

13. The method of claim 11, wherein the agent regulates a second messenger signaling pathway associated with the GPR75.

14. The method of claim 11, wherein said agent is an anti-GPR75 antibody.

15. The method of claim 14, wherein said anti-GPR75 antibody is a human or humanized monoclonal antibody.

16. A method for treating PAH comprising administering to a subject in need thereof a pharmaceutical composition comprising a therapeutically effective amount of an agent that inhibits circulating microparticles (MPs) or GRP75 expressed thereon.

17. The method of claim 16, wherein the administration of the agent reduces an amount of circulating MPs that are associated with PAH.

18. The method of claim 16, wherein the administration of the agent reduces an expression level of GPR75 expressed on MPs associated with PAH.

19. The method of claim 16, wherein the agent is an anti-GPR75 antibody.

20. The method of claim 19, wherein said anti-GPR75 antibody is a human or humanized monoclonal antibody.
